SEQUENCE LISTING (15 sequences)

SEQ ID NO: 1 - protein, 8 aa, Mus musculus
GFTFSYYW

SEQ ID NO: 2 - protein, 10 aa, Mus musculus
IRLKSDNYAT

SEQ ID NO: 3 - protein, 5 aa, Mus musculus
EGFGY

SEQ ID NO: 4 - protein, 6 aa, Mus musculus
QDVSTA

SEQ ID NO: 5 - protein, 3 aa, Mus musculus
WAS

SEQ ID NO: 6 - protein, 9 aa, Mus musculus
QQHYSTPRT

SEQ ID NO: 7 - DNA, 626 bp, Mus musculus
  CDS (184)..(525): V-D-J region of heavy chain of R-17F antibody
ctaatacgactcactatagggcaagcagtggtatcaacgcagagtacatggggctgacagaggcacctaactgtggactcacaagtctttcccttcagtgaccaacacggacacagaacattcaccatggacttgagactgagctgtgcttttattattgttcttttaaaaggggtccagagtgaagtgaagcttgaggagtctggaggagacttggtgcaacctggaggatccatgaaagtttcctgtgttgcctctggattcactttcagttattactggatgaactgggtccgccagtctccagagaaggggcttgagtgggttgctcaaattagattgaaatctgataattatgcaacacattatgcggagtctgtgaaagggaggttcaccatctcaagagatgattccagaagtagtgtctacctgcaaatgaacaacttaagggctgaagacactggaatttattactgtgaggggtttggttactggggccaagggactctggtcactgtctctgcagccaaaacgacacccccatctgtctatccactggcccctggatctgctgcccaaactaactccatggtgaccctgggatgcctggtcaagggctatttccc

SEQ ID NO: 8 - protein, 114 aa, Mus musculus
EVKLEESGGDLVQPGGSMKVSCVASGFTFSYYWMNWVRQSPEKGLEWVAQIRLKSDNYATHYAESVKGRFTISRDDSRSSVYLQMNNLRAEDTGIYYCEGFGYWGQGTLVTVSA

SEQ ID NO: 9 - DNA, 603 bp, Mus musculus
  CDS (144)..(464): V-J region of light chain of R-17F antibody
ctaatacgactcactatagggcaagcagtggtatcaacgcagagtacatgggggaaatgcatcgcaccagcatgggcatcaagatggagtcacagattcaggcatttgtattcgtgtttctctggttgtctggtgttgacggagacattgtgatgacccagtctcacaaattcatgtccacatcagtaggagacagggtcagcatcacctgcaaggccagtcaggatgtgagtactgctgtagcctggtatcaacaaaaaccagggcaatctcctaaactactgatttactgggcatccacccggcacactggagtccctgatcgcttcacaggcagtggatctgggacagattatactctcaccatcagcagtgtgcaggctgaagacctggcactttattactgtcagcaacattatagcactcctcggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtg

SEQ ID NO: 10 - protein, 107 aa, Mus musculus
DIVMTQSHKFMSTSVGDRVSITCKASQDVSTAVAWYQQKPGQSPKLLIYWASTRHTGVPDRFTGSGSGTDYTLTISSVQAEDLALYYCQQHYSTPRTFGGGTKLEIK

SEQ ID NO: 11 - DNA, 12 bp, Artificial Sequence
  Synthetic Primer
tccakagttcca

SEQ ID NO: 12 - DNA, 12 bp, Artificial Sequence
  Synthetic Primer
gctgtcctgatc

SEQ ID NO: 13 - DNA, 23 bp, Artificial Sequence
  Synthetic Primer
gggaartarcccttgaccaggca

SEQ ID NO: 14 - DNA, 23 bp, Artificial Sequence
  Synthetic Primer
gggaartagcctttgacaaggca

SEQ ID NO: 15 - DNA, 26 bp, Artificial Sequence
  Synthetic Primer
cactgccatcaatcttccacttgaca

(19) United States
(12) Patent Application Publication
(10) Pub. No.: US 2015/0344567 A1
(43) Pub. Date: Dec. 3, 2015

(54) iPS/ES CELL-SPECIFIC ANTIBODY HAVING CYTOTOXICITY TO TARGET CELLS AND USE THEREOF

(71) Applicants: THE RITSUMEIKAN TRUST, Kyoto-shi, Kyoto (JP); NATIONAL INSTITUTES OF BIOMEDICAL INNOVATION, HEALTH AND NUTRITION, Ibaraki-shi, Osaka (JP)

(72) Inventors: Toshisuke KAWASAKI, Kusatsu-shi, Shiga (JP); Nobuko KAWASAKI, Kusatsu-shi, Shiga (JP); Miho FURUE, Ibaraki-shi, Osaka (JP); Kenji KAWABATA, Ibaraki-shi, Osaka (JP); Hidenao TOYODA, Kusatsu-shi, Shiga (JP)

(21) Appl. No.: 14/654,376

(22) PCT Filed: Dec. 20, 2013
(86) PCT No.: PCT/JP2013/084374

§ 371 Date: Jun. 19, 2015

(30) Foreign Application Priority Data
Dec. 21, 2012 (JP) 2012-280259

(51) Int. Cl. C07K 16/28; A61K 39/395; A61K 35/545; G01N 33/569

(52) U.S. Cl. CPC C07K 16/28; G01N 33/56966; A61K 39/39533; A61K 35/545; C07K 2317/565; C07K 2317/56; A61K 2039/507

(57) ABSTRACT

The present invention provides a monoclonal antibody that recognizes a lipid substance on an iPS cell surface and an ES cell surface as an epitope, and does not recognize EC cells, the antibody having a cytotoxic activity against a target cell, a method of producing a uniform differentiated cell population free of an undifferentiated cell, including contacting a cell population differentiated from an iPS or ES cell with the above antibody, and recovering viable cells, an agent for a cell transplantation therapy, containing a differentiated cell population obtained by the method, and the like.

### TECHNICAL FIELD

The present invention relates to a monoclonal antibody that specifically binds to an induced pluripotent stem cell (iPS cell) and an embryonic stem cell (ES cell), and has a cytotoxic activity against the target cells, and use thereof. More particularly, the present invention relates to a monoclonal antibody that recognizes a lipid substance on an iPS/ES cell surface, which is different from those recognized by known anti-iPS/ES cell antibodies, as well as use of said antibody as a marker antibody of human iPS/ES cells and a cytotoxic agent for selective elimination of the cells.

### BACKGROUND ART

The establishment of human induced pluripotent stem cell (iPS cell) has opened the door to the practicalization of a cell transplantation treatment using a pluripotent stem cell. For example, in the case of a chronic disease such as Parkinson's disease and Type I diabetes mellitus, if an iPS cell can be established from the patient, induced to differentiate into a necessary cell, and autologously transplanted to the patient, the ethical issue associated with the use of a human embryonic stem cell (ES cell) (namely, destruction of early embryo which may be the emerging potential of human. life) and the problem of rejection in transplantation can be avoided. On the other hand, since it takes at least 2-3 months from the establishment of an iPS cell to differentiation induction into the object cell, iPS cells of various HLA types or differentiated cells derived therefrom may be banked and used for allogenic transplantation in the case of a disease requiring early treatments such as spinal damage, fulminant hepatitis and the like.

However, when pluripotent stem cells such as ES cell, iPS cell and the like are cultivated under conditions for differentiation into cells of cardiac muscle, nerve and the like, undifferentiated cells remain in the differentiated cell population to cause tumorization (teratoma, carcinogenesis). Furthermore, since iPS cell is artificially reprogrammed, it also has unique safety problems (i.e., tumorization risk due to the introduction of protooncogenes such as c-Myc and the like and the use of virus vector, tumorization risk due to resistance to differentiation depending on the kind of somatic cells to be the derivation and the like).

Thus, it is essential for the practicalization of a regenerative transplantation treatment using pluripotent stem cells to overcome the problem of tumorization. To suppress carcinogenesis derived from iPS cell, various attempts have been made from the aspects of establishment of safer iPS cell, such as search for a combination of reprogramming factors free of oncogene, use of nonvirus vector, establishment of iPS cell by protein introduction and the like. Nevertheless, they are only indirect approaches that suppress carcinogenesis risk somewhat by working on iPS production, which is not of a level capable of completely preventing carcinogenesis.

In addition, an effective solving means for the risk of tumorization (teratoma in which various kinds of cells other than the object cells are mixed to form a mass) due to the remaining undifferentiated cells since the pluripotent stem cell is also common to ES cells, has not been provided.

In the meantime, a sugar chain-recognizing antibody is a probe that sharply perceives changes in the cellular surface sugar chain, and is also widely utilized as a marker antibody of human iPS/ES cells. That is, an epitope of SSEA3, SSEA4 is a glycolipid of the globo-series, and the epitope of TRA-1-60, TRA-1-81 is one kind of keratansulfuric acid. However, most of these existing antibodies were in fact obtained by using EC cells (embryonal carcinoma cell) as an immunogen, and also react with EC cells (cancer cells) besides iPS/ES cells (non-patent document 1).

In the study of stem cells and regenerative medicine, therefore, emergence of an antibody that does not react with EC cells but reacts with iPS/ES cells alone has been awaited. Recently, Choo reported an anti-human ES cell antibody (mAb84) that does not react with EC cells, by using human ES cell as an immunogen (patent document 1). However, it is not described whether the antibody also reacts with human iPS cell (patent document 2).

The present inventors successfully acquired an iPS/ES cell positive and EC cell negative antibody (R-10G) by immunizing a mouse with human iPS cell (Tic) as an immunogen, and subjecting the obtained hybridoma to differential screening by human iPS cells and human EC cells (patent document 2). This antibody recognizes keratansulfuric acid different from epitopes of TRA-1-60 and TRA-1-81 bound to podocalyxin protein on iPS/ES cell surface.

However, since R-10G does not have a cytotoxic activity against human iPS/ES cells, use of said antibody for the elimination of human iPS/ES cells remaining in differentiated cell population requires a separation operation using flow cytometry or affinity carrier.

### DOCUMENT LIST

### Patent Documents

patent document 1: WO 2007/102787
patent document 2: WO 2012/147992

### Non-Patent Document

non-patent document 1: Wright, A. J. and Andrews, P. W., Stem Cell Res., 3(1): 3-11 (2009)

### SUMMARY OF THE INVENTION

### Problems to be Solved by the Invention

An object of the present invention is to provide a novel anti-iPS/ES means having a cytotoxic activity specific to a target cell and capable of selectively eliminating, by targeting and killing, undifferentiated cells remaining in a cell population that has been induced to differentiate from iPS/ES cells and causing post-transplantation tumorization, thereby providing safe transplantation cells free from a tumorization risk and differentiated cells highly reliable as an efficacy, toxicity evaluation system, and find a way of practicalization of a cell transplantation treatment using a stem cell and progress of drug discovery and development.

### Means of Solving the Problems

The present inventors have conducted intensive studies in an attempt to solve the aforementioned problems, isolated a different human iPS/ES cell-positive and human EC cell-negative monoclonal antibody (named R-17F), which is considered to recognize a lipid substance, by using a method similar to that for R-10G antibody, and found that the antibody unexpectedly shows a strong, antibody concentration-dependent complement-independent cytotoxic action on human iPS cells. Such action was markedly enhanced by the addition of a trace amount of a secondary antibody.

The present inventors have conducted further studies based on these findings and completed the present invention.

Accordingly, the present invention is as described below.
[1] A monoclonal antibody that recognizes a lipid substance on an iPS cell surface and an ES cell surface as an epitope, and does not recognize EC cells.
[2] The antibody of the above-mentioned [1], wherein the iPS and ES cells are derived from human.
[3] The antibody of the above-mentioned [1] or [2], which is a monoclonal antibody produced by hybridoma R-17F (accession number: NITE BP-01425), or a monoclonal antibody that recognizes, as an epitope, a region same as a region of a lipid substance recognized by the monoclonal antibody produced by hybridoma R-17F.
[4] The antibody of any of the above-mentioned [1]-[3], wherein the lipid substance is a glycolipid and the aforementioned region comprises a sugar chain represented by the following formula:

Fuc-Hex-HexNAc-Hex-Hex

wherein Fuc is fucose, Hex is hexose, and HexNAc is N-acetylhexosamine.
[5] The antibody of the above-mentioned [4], which recognizes, as an epitope, at least a region comprising a sugar chain represented by the following formula:

Fuc(α1-2)Gal(β1-3)GlcNAc(β1-3)Gal(β1-4)Glc

wherein Fuc is fucose, Gal is galactose, GlcNAc is N-acetylglucosamine, and Glc is glucose, in the glycolipid.
[6] The antibody of any of the above-mentioned [1]-[5], comprising
(a) CDR comprising the amino acid sequence shown in SEQ ID NO: 1,
(b) CDR comprising the amino acid sequence shown in SEQ ID NO: 2,
(c) CDR comprising the amino acid sequence shown in SEQ ID NO: 3,
(d) CDR comprising the amino acid sequence shown in SEQ ID NO: 4,
(e) CDR comprising the amino acid sequence shown in SEQ ID NO: 5, and
(f) CDR comprising the amino acid sequence shown in SEQ ID NO: 6.
[7] The antibody of the above-mentioned [6], comprising (1) a heavy chain variable region comprising the amino acid sequence shown in SEQ ID NO: 8, and
(2) a light chain variable region comprising the amino acid sequence shown in SEQ ID NO: 10.
[8] The antibody of any of the above-mentioned [1]-[7], which has a cytotoxic activity against a target cell.
[9] A reagent for detecting an iPS or ES cell, comprising the antibody of any of the above-mentioned [1]-[8].
[10] A method of detecting an iPS or ES cell, comprising contacting a cell sample with the antibody of any of the above-mentioned [1]-[8], and detecting a cell bound to the antibody in the sample.
[11] An agent for eliminating an iPS or ES cell, comprising the antibody of any of the above-mentioned [1]-[8].
[12] The agent of the above-mentioned [11], further comprising a secondary antibody to the aforementioned antibody.
[13] A method of eliminating an iPS or ES cell in a cell population, comprising contacting the cell population with the antibody of any of the above-mentioned [1]-[8].
[14] The method of the above-mentioned [13], comprising further contacting the cell population with a secondary antibody to the aforementioned antibody.
[15] A method of producing a uniform differentiated cell population free of an undifferentiated cell, comprising contacting a cell population differentiated from an iPS or ES cell with the antibody of any of the above-mentioned [1]-[8], and recovering viable cells.
[16] The method of the above-mentioned [15], comprising further contacting the cell population differentiated from the aforementioned iPS or ES cell with a secondary antibody to the aforementioned antibody.
[17] An agent for a cell transplantation therapy, comprising a cell population differentiated from iPS or ES cells and the antibody of any of the above-mentioned [1]-[8] in combination.
[18] An agent for a cell transplantation therapy, comprising a differentiated cell population obtained by the method of the above-mentioned [15] or [16].

### Effect of the Invention

Since the anti-iPS/ES cell antibody of the present invention has a specific cytotoxic activity against target iPS/ES cells, it can selectively kill and eliminate undifferentiated cells remaining in a differentiated cell population induced from human iPS/ES cells, and can provide safe cells for transplantation, which are free of a carcinogenic risk. Moreover, since the anti-iPS/ES cell antibody of the present invention does not recognize EC cells, normal growth and abnormal growth of pluripotent stem cells can be distinguished using said antibody.

### BRIEF DESCRIPTION OF THE DRAWINGS

FIG. 1 is a Figure showing protein staining of (A) Tic cell extract. A cell lysate (15 μg protein) obtained by treating Tic cells with complete RIPA buffer was dissolved in SDS-PAGE buffer, electrophoresed by using 4-15% gel plate under non-reducing conditions, and Gel code blue staining was performed. Lane M: molecular weight marker; Cell lysate: Tic cell extract. (B) is a Figure showing the results of Western blot analysis of culture supernatant of hybridoma. The gel after electrophoresis was blotted on PVDF membrane, and immunostained using culture supernatant of each hybridoma as the source of antibodies. The number on top of the lane shows hybridoma number. Lane M: molecular weight marker.

FIG. 2 is a Figure showing the results of identification of localization of R-17F, SSEA-3, SSEA-4 epitopes on iPS cell surface by a laser confocal microscope. The upper panels show the results of immunostaining of Tic cells with each antibody. Nomarski: differential interference microscope image. The lower panels show enlarged images (×80) of double staining with SSEA-4 and SSEA3 (left), and triple staining with SSEA-4, SSEA-3 and R-17F (right).

FIG. 3 is a Figure showing a concentration-dependent cytotoxic activity of R-17F antibody against human iPS cells. 17F: R-17F antibody treatment; α-MBP: anti-α-mannan-binding protein antibody treatment. The antibody concentration is shown by an antibody amount (μg) per 0.1 mL of the reaction mixture.

FIG. 4 is a Figure showing the analysis results of temperature dependency of the cytotoxic activity of R-17F antibody against human iPS cells. 17F: R-17F antibody treatment; α-MBP: anti-α-mannan-binding protein antibody treatment.

FIG. 5 is a Figure showing the reaction time-dependent cytotoxic activity of R-17F antibody against human iPS cells. 17F: R-17F antibody treatment; α-MBP: anti-α-mannan-binding protein antibody treatment.

FIG. 6 is a Figure showing an action of a secondary antibody to enhance cytotoxic activity of R-17F antibody against human iPS cells. 17F: R-17F antibody treatment; α-MBP: anti-α-mannan-binding protein antibody treatment. The secondary antibody concentration is shown by an antibody amount (μg) per 0.1 mL of the reaction mixture.

FIG. 7 is a Figure showing comparison of iPS cells cytotoxic activity of R-17F antibody with that of known anti-iPS/ES cell antibody. The left panel shows comparison with R-10G antibody. The bar graph shows the results of anti-α-mannan-binding protein antibody treatment (control), R-17F antibody treatment, and R-10G antibody treatment from the left. The right panel shows comparison with TRA-1-60, TRA-1-81 and SSEA-4. The bar graph shows the results of anti-α-mannan-binding protein antibody treatment (control), R-17F antibody treatment, TRA-1-60 treatment, TRA-1-81 treatment, and SSEA-4 treatment from the left.

FIG. 8 is a Figure showing that the iPS cells cytotoxic activity by R-17F antibody is effective even when cells form colonies and are growing. The columns on the left side show observation of progress of growth time without addition of an antibody under a phase contrast microscope. The center columns show the results of culture for 72 hr with addition of R-10G antibody, and the right columns show the results with addition of R-17F.

FIG. 9 is a Figure showing binding of R-17F antibody to iPS cells (Tic & 201B7) and ES cells (H9 & KhES-3). Respective cells were reacted with R-17F antibody, then with fluorescence-labeled secondary antibody, and cell bindability was measured by a flow cytometer.

FIG. 10 is a Figure showing the cytotoxic activity of R-17F antibody against iPS cells (Tic & 201B7) and ES cells (H9 & KhES-3), and concentration dependency thereof.

FIG. 11 is a Figure showing that the epitope of R-17F is a glycolipid. (A) shows the effect of glycolipid synthesis inhibitor PDMP treatment on the reactivity of R-17F antibody with Tic cells. (B) shows the analysis results of glycolipidic R-17F epitope by TLC-immunostaining. Total lipid components were extracted from Tic cells, separated by TLC, and stained by primuline (L) and immunostained by R-17F. The results thereof (R) are shown. (C) shows the results of MALDI-TOF MS analysis of band [A] separated and purified by TLC.

FIG. 12 is a Figure showing that R-17F antibody selectively binds to Lacto-N-fucopentaose I (LNFP I).

FIG. 13 is a Figure showing (A) comparison of reactivity of R-17F antibody (upper panel) and that of known antibody mAb84 (lower panel) with Tic cells, and (B) cytotoxic activity of mAb84 against Tic cells.

### DESCRIPTION OF EMBODIMENTS

### [I] Antibody of the Present Invention

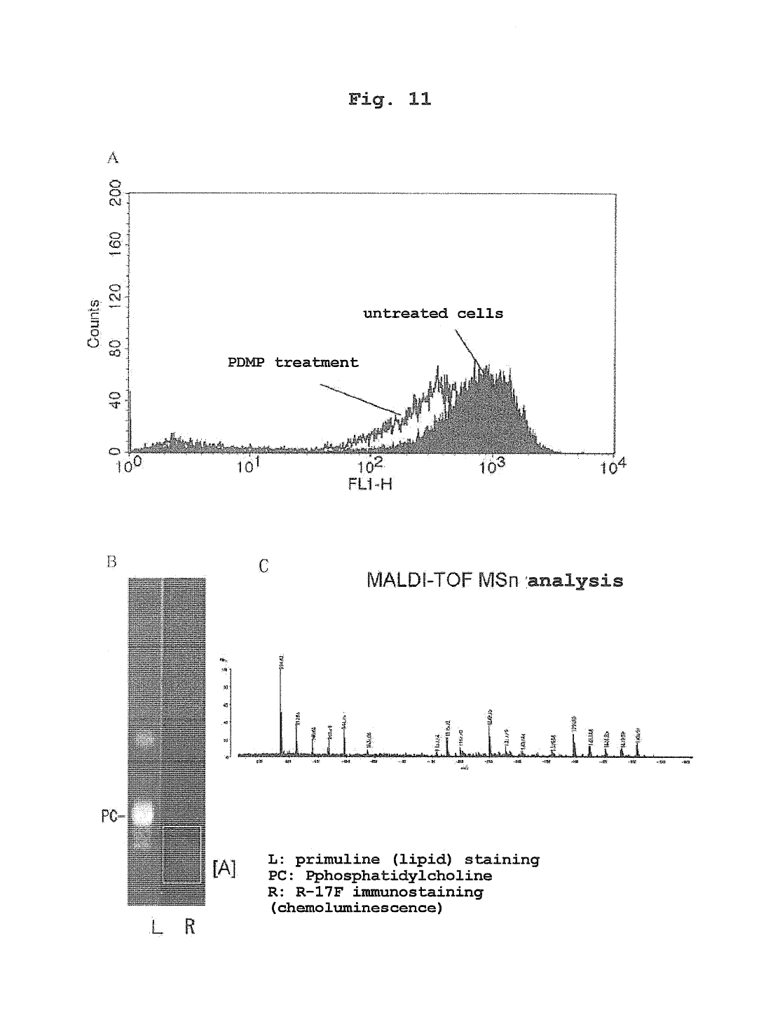

The present invention provides a monoclonal antibody capable of specifically recognizing iPS and ES cells (hereinafter sometimes to be referred to as “the anti-iPS/ES cell antibody of the present invention” or simply as “the antibody of the present invention”). The antibody is further characterized by (a) not recognizing EC cells, and (b) recognizing a lipid substance present on a surface of iPS and ES cells, more specifically, a glycolipid. Since known anti-iPS/ES cell antibodies that recognize glycolipids SSEA-3, SSEA-4 and the like also recognize EC cells, the anti-iPS/ES cell antibody of the present invention was considered to recognize the structure of a lipid molecule different from those of the glycolipids recognized by known antibodies. In fact, in Far-eastern blotting of all lipid components of human iPS cell by using R-17F antibody and SSEA-4 antibody, each antibody recognized different lipid substances (see FIG. 11B, data relating to SSEA-4 antibody was omitted). Moreover, since the reactivity of R-17F antibody with human iPS cell decreases by inhibiting an enzyme reaction that converts ceramide to glucosylceramide, which is a starting material of ganglioside series or globoside series glycolipid biosynthesis (see FIG. 11A), it was suggested that a lipid molecule recognized by said antibody is a glycolipid using glucosylceramide as a starting material.

That an anti-iPS/ES cell antibody recognizes a lipid substance on iPS and ES cell surface can be confirmed by, for example, extracting lipid components from a cellular membrane of iPS or ES cell with an organic solvent and the like, separating the lipid by, for example, thin layer chromatography (TLC) and the like, and immunostaining (Far-eastern blotting) same with the anti-iPS/ES cell antibody. It is also possible to identify a lipid substance recognized by said antibody by isolating lipids that reacted with said antibody and subjecting same to mass spectrometry and NMR analysis.

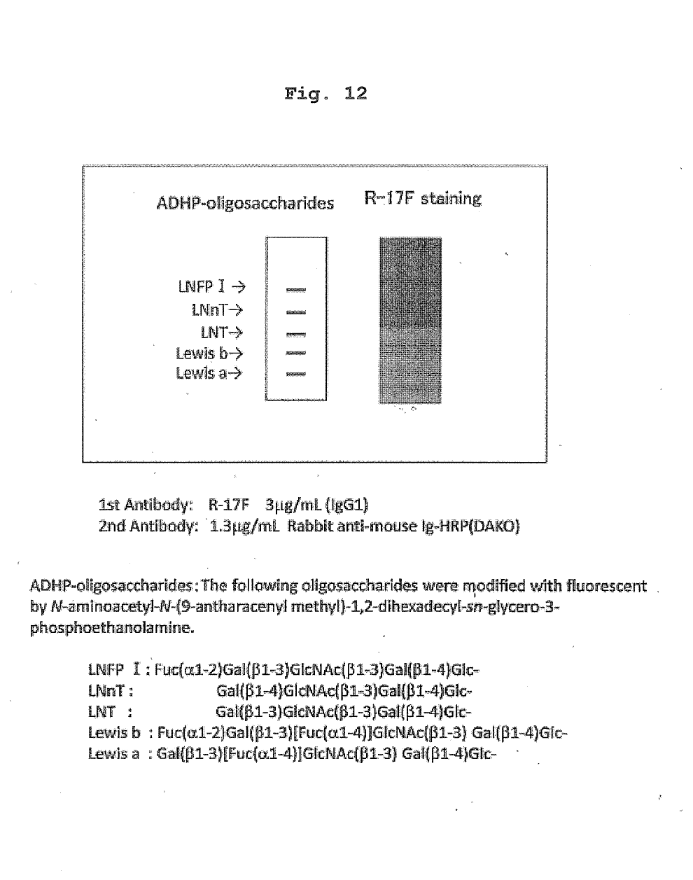

In the aforementioned Far-eastern blotting (FIG. 11B), TLC band bound to R-17F antibody was analyzed by MALDI-TOF MS (see FIG. 11C) and tandem MS. As a result, R-17F antibody was shown to recognize and bind to Fuc-Hex-HexNAc-Hex-Hex-ceramide (Fuc: fucose, Hex: hexose, HexNAc: N-acetylhexosamine). Furthermore, the binding activity of a synthetic lipid containing lacto series and neolacto series sugar chain and R-17F antibody was examined based on the structural analysis results. As a result, R-17F antibody bound to a lipid containing Lacto-N-fucopentaose I (Fuc(α1-2)Gal(β1-3)GlcNAc(β1-3)Gal(β1-4)Glc; Fuc: fucose, Gal: galactosamine, GlcNAc: N-acetylglucosamine, Glc: glucose) (sometimes to be abbreviated as “LNFP I” in the present specification), but did not bind to a lipid containing Lacto-N-tetraose and Lacto-N-neotetraose free of fucose on the terminal, or branched Lewis a or Lewis b sugar chain (FIG. 12).

Therefore, in a preferable embodiment, the antibody of the present invention is characterized in that it recognizes a glycolipid containing linear pentaose having fucose on the terminal, i.e., Fuc-Hex-HexNAc-Hex-Hex, preferably sphingoglycolipid containing LNFP I or Fuc(α1-2)Gal(β1-4)GlcNAc(β1-3)Gal(β1-4)Glc, which is a neolacto series sugar chain corresponding thereto, as an epitope.

The antibody of the present invention may or may not have a cytotoxic activity against the target iPS/ES cells. In a preferable embodiment, the antibody of the present invention has a cytotoxic activity at least against iPS cells, more preferably also has a cytotoxic activity against ES cells. The cytotoxic activity may be based on any mechanism and examples thereof include, but are not limited to, antibody dependent cytotoxic activity (ADCC), complement dependent cytotoxic activity (CDC), antibody dependent phagocytosis (ADCP), ADCC/CDC independent apoptosis/necrosis induction action and the like. The anti-iPS/ES cell antibody R-17F described in the below-mentioned Examples proceeds temperature independently, and shows a cytotoxic activity under culture conditions free of complement components, which shows that it has a complement independent cytotoxic activity. The cell death by R-17F antibody is like necrosis.

Whether an anti-iPS/ES cell antibody has a cytotoxic activity against a target cell can be examined by a method known per se (e.g., WO 2007/102787). Those of ordinary skill in the art can select either a cytotoxic antibody or a non-cytotoxic antibody according to the use object of the antibody.

In one preferable embodiment, the antibody of the present invention is an R-17F antibody or an antibody having the same complementarity determining region (CDR).

The basic structure of the antibody molecule is common to all classes, and constituted of a heavy chain having a molecular weight of 50,000-70,000 and a light chain having a molecular weight of 20,000-30,000 (Immunology Illustrated (ed. I. Roitt, J. Brostoff, D. Male)). A heavy chain is generally composed of a polypeptide chain containing about 440 amino acids, has a characteristic structure for each class, and is called γ, μ, α, δ, ε chain corresponding to IgG, IgM, IgA, IgD, IgE. Furthermore, IgG includes IgG1, IgG2, IgG3, IgG4, which are called γ1, γ2, γ3, γ4, respectively. A light chain is generally composed of a polypeptide chain containing about 220 amino acids, and two kinds of L type and K type, called A, chain, respectively, are known. The peptide constitution of the basic structure of the antibody molecule contains two heavy chains and two light chains homologous to each other and bonded by a disulfide bond (S—S bond) and a noncovalent bond, and has a molecular weight of 150,000-190,000. The two kinds of light chains can make a pair with any heavy chain. Each antibody molecule is always composed of the same two light chains and the same two heavy chains.

A heavy chain contains 4 (5 in μ, ε chains) S—S bonds, and a light chain contains 2 S—S bonds, thereby forming one loop per 100-110 amino acid residues. The steric structure is similar between loops, and called a structure unit or domain. The domain present at the N terminal of heavy chain and light chain has a varying amino acid sequence even in a reference standard from the same class (subclass) of animals of the same species and is called a variable region (V region) (heavy chain variable region domain is indicated as V_{H}, light chain variable region domain is indicated as V_{L}). Thus, the amino acid sequence on the C-terminal side is almost constant for each class or subclass and is called a constant region (C region) (each domain is indicated as C_{H}1, C_{H}2, C_{H}3 or C_{L}).

An antigen binding site of an antibody is constituted of V_{H} and V_{L}, and the specificity of the binding is based on the amino acid sequence of the site. On the other hand, biological activity such as binding with complement and various cells reflects structural difference among C regions of class Igs. It has been clarified that the variability of variable regions of light chain and heavy chain is mostly limited to three small hypervariable regions present in both chains, and these regions are called complementarity determining region (CDR). Of the variable region, the part other than CDR is called a framework region (FR), and is comparatively constant. The framework region adopts β sheet conformation and CDR can form a loop that connects β sheet structures. CDR in each chain is maintained in the tertiary structure thereof by a framework region and forms an antigen binding site together with CDR from other chain.

Some numbering systems are conventionally used to identify CDR. Kabat definition is based on the sequence variability, and Chothia definition is based on the position of structural loop region. AbM definition is between Kabat and Chothia approaches. The boundary of CDRs of variable regions of light chain and heavy chain is shown according to the Kabat, Chothia or AbM algorithm (Martin et al. (1989) Proc. Natl. Acad. Sci. USA 86: 9268-9272; Martin et al. (1991) Methods Enzymol. 203: 121-153; Pedersen et al. (1992) Immunomethods 1: 126; and Rees et al. (1996) In Sternberg M.J.E. (ed.), Protein Structure Prediction, Oxford University Press, Oxford, pp. 141-172).

CDR of the antibody of the present invention is defined to be a CDR identified by analyzing the nucleotide sequences of the variable regions (V_{H} and V_{L}) of heavy chain and light chain genes of said antibody, by using IMGT/V-QUEST (http://www.imgt.org/IMGT_vquest/share/textes/) which is an integrated system for standardized analysis of reconstituted nucleotide sequences of immunoglobulin and T cell receptor, provided by University of Montpellier 2.

In the case of R-17F antibody, CDR of the heavy chain variable region includes amino acid Nos 26-33 (CDR1-H), 51-60 (CDR2-H) and 99-103 (CDR3-H) of the amino acid sequence shown in SEQ ID NO: 8, and CDR of the light chain variable region includes amino acid Nos 27-32 (CDR1-L), 50-52 (CDR2-L) and 89-97 (CDR3-L) of the amino acid sequence shown in SEQ ID NO: 10

Therefore, in one preferable embodiment, the antibody of the present invention is
(1) an antibody containing
   (a) CDR comprising the amino acid sequence shown by Gly Phe Thr Phe Ser. Tyr Tyr Trp (SEQ ID NO: 1),
   (b) CDR comprising the amino acid sequence shown by Ile Arg Leu Lys Ser Asp Asn Tyr Ala Thr (SEQ ID NO: 2),
   (c) CDR comprising the amino acid sequence shown by Glu Gly Phe Gly Tyr (SEQ ID NO: 3),
   (d) CDR comprising the amino acid sequence shown by Gln Asp Val Ser Thr Ala (SEQ ID NO: 4),
   (e) CDR comprising the amino acid sequence shown by Trp Ala Ser (SEQ ID NO: 5), and
   (f) CDR comprising the amino acid sequence shown by Gln Gln His Tyr Ser Thr Pro Arg Thr (SEQ ID NO: 6), or
(2) an antibody containing CDRs of the above-mentioned (a)-(f), having one or more (e.g., 1, 2, 3, 4, 5 or 6) amino acid sequences selected from the amino acid sequences shown in SEQ ID NOs: 1-6, wherein one or two amino acid residues are substituted and/or deleted and/or added and/or inserted in each sequence, which specifically recognizes iPS and ES cells but does not recognize EC cells.

More preferably,
(1) an antibody containing a light chain variable region containing CDRs of the above-mentioned (a)-(c), and a heavy chain variable region containing CDRs of the above-mentioned (d)-(f), or
(2) an antibody containing the light chain and heavy chain variable regions of the above-mentioned (1), having one or more (e.g., 1, 2, 3, 4, 5 or 6) amino acid sequences selected from the amino acid sequences shown in SEQ ID NOs: 1-6, wherein one or two amino acid residues are substituted and/or deleted and/or added and/or inserted in each sequence, which specifically recognizes iPS and ES cells but does not recognize EC cells.

More preferably, in the above-mentioned antibody, CDRs of (a), (b) and (c) are set in this order from the N terminal of the light chain. That is, CDRs of (a), (b) and (c) correspond to CDR1, CDR2 and CDR3 of the heavy chain, respectively. Similarly, CDRs of (d), (e) and (f) are set in this order from the N terminal of the heavy chain. That is, CDRs of (d), (e) and (f) correspond to CDR1, CDR2 and CDR3 of the light chain, respectively.

A still more preferable example of the antibody of the present invention is
(1) an antibody containing a heavy chain variable region containing the amino acid sequence shown in SEQ ID NO: 8 and a light chain variable region containing the amino acid sequence shown in SEQ ID NO: 10, or
(2) an antibody containing the light chain and heavy chain variable regions of the above-mentioned (1), having either one or both of SEQ ID NOs: 8 and 10, wherein one or more, preferably 1-20, more preferably 1-10, further preferably 1-several (e.g., 1, 2, 3, 4 or 5), amino acid residues are substituted and/or deleted and/or added and/or inserted in each sequence, which specifically recognizes iPS and ES cells but does not recognize EC cells.

While the isotype of the antibody of the present invention is not particularly limited, it is preferably IgG, IgM or IgA, particularly preferably IgG.

The antibody of the present invention is not subject to limitation on the form of molecules as long as it has at least a complementarity determining region (CDR) for specifically recognizing and binding to the antigenic determinant (epitope); in addition to the whole antibody molecule, the antibody may, for example, be a fragment such as Fab, Fab′, or F(ab′)₂, a genetically engineered conjugate molecule such as scFv, scFv-Fc, minibody, or diabody, or a derivative thereof modified with a molecule having protein stabilizing action, such as polyethylene glycol (PEG), or the like, and the like.

### [II] Production of the Antibody of the Present Invention

The antibody of the present invention can be produced by a method of antibody production known per se. Hereinafter, a method of preparing an immunogen (iPS/ES cell) for producing the antibody of the present invention, and a method of producing the antibody are described.

### (1) Preparation of Immunogen

As an antigen used to prepare the antibody of the present invention, iPS and ES cells or a fraction thereof (e.g., membrane fraction) containing a lipid substance on a cell surface and the like can be used.

An iPS cell can be produced by reprogramming a somatic cell obtained from a mammal according to any methods [see, for example, Cell 2007; 131:861-72, Science 2007; 318:1917-20 (human); Cell 2006; 126:663-76 (mouse); Cell Stem Cell 2008; 3(6):587-90 (Rhesus monkey); Cell Stem Cell 2008; (1):11-5, Cell Stem Cell 2008; 4(1):16-9 (rat); J Mol Cell Biol 2009; 1(1):6-54 (pig); Mol Reprod Dev 2010; 77(1):2 (dog); Stem Cell Res 2010; 4(3):180-8, Genes Cells 2010; 15(9):959-69 (marmoset); J Biol Chem 2010; 285(41):31362-9 (rabbit)].

Also, iPS cells can be obtained from various public and private depositories and are commercially available. For example, human iPS cell lines 201B7 and 235G1 can be obtained from CELL BANK of RIKEN BIORESOURCE CENTER and Tic (JCRB1331) can be obtained from National Institute of Biomedical Innovation.

An ES cell can be produced by any known methods. For example, available methods of preparing ES cells include, but are not limited to, methods in which an inner cell mass is dissected from the embryo of a mammal in the blastocyst stage and cultured [see, for example, Manipulating the Mouse Embryo: A Laboratory Manual, Second Edition, Cold Spring Harbor Laboratory Press (1994)] and methods in which an early embryo prepared by somatic cell nuclear transfer is cultured (Nature 1997; 385:810, Science 1998; 280:1256, Protein Nucleic Acid and Enzyme 1999; 4:892, Nat Biotechnol 1999; 17:456, Nature 1998; 394:369, Nat Genet 1999; 22:127, Proc Natl Acad Sci USA 1999; 96:14984, Nat Genet 2000; 24:109).

Also, ES cells can be obtained from various public and private depositories and are commercially available. For example, human ES cell lines H1 and H9 can be obtained from the cell bank of WiCell Institute at the University of Wisconsin and KhES-1, -2 and -3 can be obtained from the cell bank of Institute for Frontier Medical Sciences, Kyoto University or RIKEN BioResource Center.

Intact iPS or ES cells may be used for immunization, or freeze-thawed, irradiated or glutaraldehyde-treated iPS or ES cells also may be used.

Alternatively, a cell membrane fraction of the iPS or ES cells can be used as an immunogen for producing the antibody of the present invention. The cell membrane fraction can be prepared by homogenizing iPS or ES cells, removing the cell debris by low speed centrifugation, thereafter precipitating a cell membrane-containing fraction by high speed centrifugation of the supernatant (and, where necessary, purifying the cell membrane fraction by density gradient centrifugation and the like).

### (2) Production of Monoclonal Antibody

### (a) Production of Monoclonal Antibody-Producing Cells

The immunogen prepared as mentioned above is administered as is, or along with a carrier or a diluent, to a warm-blooded animal at a site enabling antibody production by the methods such as intraperitoneal injection, intravenous injection, subcutaneous injection, intradermal injection and the like. In order to increase antibody productivity upon the administration, Freund's complete adjuvant or Freund's incomplete adjuvant may be administered. Dosing is normally performed about 2 to 10 times in total every 1 to 6 weeks. As examples of the warm-blooded animal to be used, mouse, rat rabbit, goat, monkey, dog, guinea pig, sheep, donkey and chicken, preferably mouse, rat and rabbit can be mentioned.

Alternatively, the immunogen can be subjected to in vitro immunization method. As the animal cells used in the in vitro immunization method, lymphocytes, preferably B-lymphocytes and the like, isolated from peripheral blood, spleen, lymph node and the like of a human and the above-described warm-blooded animals (preferably mouse or rat) can be mentioned. For example, in the case of mouse or rat cells, the spleen is extirpated from an about 4- to 12-week-old animal, and splenocytes are separated and rinsed with a appropriate medium [e.g., Dulbecco's modified Eagle medium (DMEM), RPMI1640 medium, Ham's F12 medium and the like], after which the splenocytes are suspended in an antigen-containing medium supplemented with fetal calf serum (FCS; about 5 to 20%) and cultured using a CO₂ incubator and the like for about 4 to 10 days. Examples of the antigen concentration include, but are not limited to, 0.05-5 μg. It is preferable to prepare a culture supernatant of thymocytes of an animal of the same strain (preferably at about 1 to 2 weeks of age) according to a conventional method, and to add the supernatant to the medium.

Because it is difficult to obtain a thymocyte culture supernatant in in vitro immunization of human cells, it is preferable to perform immunization by adding, to the medium, several kinds of cytokines such as IL-2, IL-4, IL-5, and IL-6 and the like, and if necessary, an adjuvant substance (e.g., muramyldipeptide and the like) along with the antigen.

In preparing a monoclonal antibody, it is possible to establish an antibody-producing hybridoma by selecting an individual or cell population showing an elevated antibody titer from among antigen-immunized warm-blooded animals (e.g., mice, rats) or animal cells (e.g., human, mouse, rat), respectively; collecting spleens or lymph nodes at 2 to 5 days after the final immunization or collecting the cells after 4 to 10 days of cultivation after in vitro immunization to isolate antibody-producing cells; and fusing the isolated cells with myeloma cells. A measurement of serum antibody titer can be performed by, for example, reacting a labeled antigen and an antiserum, and thereafter determining the activity of the label bound to the antibody.

Although the myeloma cells are not subject to limitation, as long as they are capable of producing a hybridoma that secretes a large amount of antibody, those that do not produce or secrete the antibody per se are preferable, with greater preference given to those of high cell fusion efficiency. To facilitate hybridoma selection, it is preferable to use a cell line that is susceptible to HAT (hypoxanthine, aminopterin, thymidine). As examples of the mouse myeloma cells, NS-1, P3U1, SP2/0, AP-1 and the like can be mentioned; as examples of the rat myeloma cells, R210.RCY3, Y3-Ag 1.2.3 and the like can be mentioned; as examples of the human myeloma cells, SKO-007, GM 1500-6TG-2, LICR-LON-HMy2, UC729-6 and the like can be mentioned.

Fusion operation can be performed according to a known method, for example, the method of Koehler and Milstein [Nature, 256, 495 (1975)]. As a fusion promoter, polyethylene glycol (PEG), Sendai virus and the like can be mentioned, and PEG and the like are preferably used. Although the molecular weight of PEG is not subject to limitation, PEG1000 to PEG6000, which are of low toxicity and relatively low viscosity, are preferable. As examples of the PEG concentration, about 10 to 80%, preferably about 30 to 50%, can be mentioned. As the solution for diluting PEG, various buffers such as serum-free medium (e.g., RPMI1640), complete medium comprising about 5 to 20% serum, phosphate buffered saline (PBS), and Tris buffer can be used. DMSO (e.g., about 10 to 20%) can also be added as desired. As examples of the pH of the fusion solution, about 4 to 10, preferably about 6 to 8 can be mentioned.

The ratio by number of antibody-producing cells (splenocytes) and myeloma cells is preferably about 1:1 to 20:1, and the cell fusion can be efficiently performed by incubation normally at 20 to 40° C., preferably at 30 to 37° C., normally for 1 to 10 minutes.

An antibody-producing cell line can also be obtained by infecting antibody-producing cells with a virus capable of transforming lymphocytes to immortalize the cells. As such viruses, for example, Epstein-Barr (EB) virus and the like can be mentioned. Although the majority of persons have immunity because they have ever been infected with this virus in an asymptomatic infection of infectious mononucleosis, virion is also produced when the ordinary EB virus is used; therefore, appropriate purification must be performed. As an EB system free from the possibility of viral contamination, it is also preferable to use a recombinant EB virus that retains the capability of immortalizing B lymphocytes but lacks the capability of replicating virion (for example, deficiency of the switch gene for transition from latent infection state to lytic infection state and the like).

Since marmoset-derived B95-8 cells secrete EB virus, B lymphocytes can be easily transformed by using a culture supernatant thereof. An antibody-producing B cell line can be obtained by, for example, culturing these cells using a medium supplemented with serum and penicillin/streptomycin (P/S) (e.g., RPMI1640) or a serum-free medium supplemented with a cell growth factor, thereafter separating the culture supernatant by filtration or centrifugation and the like, suspending therein antibody-producing B lymphocytes at a suitable concentration (e.g., about 10⁷ cells/mL), and incubating the suspension normally at 20 to 40° C., preferably at 30 to 37° C., normally for about 0.5 to 2 hours. When human antibody-producing cells are provided as mixed lymphocytes, it is preferable to previously remove T lymphocytes by allowing them to form an E rosette with, for example, sheep erythrocytes and the like, to increase transformation frequency of EB virus, because the majority of persons have T lymphocytes which exhibit cytotoxicity to cells infected with EB virus. It is also possible to select lymphocytes specific for the target antigen by mixing sheep erythrocytes, previously bound to a soluble antigen, with antibody-producing B lymphocytes, and separating the rosette using a density gradient of percoll and the like. Furthermore, because antigen-specific B lymphocytes are capped by adding the antigen in large excess so that they no longer present IgG to the surface, mixing with sheep erythrocytes bound to anti-IgG antibody results in the formation of rosette only by antigen-nonspecific B lymphocytes. Therefore, by collecting a layer of cells that don't form rosette from this mixture using a density gradient of percoll and the like, it is possible to select antigen-specific B lymphocytes.

Human antibody-secreting cells having acquired the capability of proliferating indefinitely by the transformation can be back fused with mouse or human myeloma cells in order to stably sustain the antibody-secreting ability. As the myeloma cells, the same as those described above can be used.

Hybridoma screening and breeding are normally performed using a medium for animal cells (e.g., RPMI1640) containing 5 to 20% FCS or a serum-free medium supplemented with cell growth factors, with the addition of HAT (hypoxanthine, aminopterin, thymidine). As examples of the concentrations of hypoxanthine, aminopterin and thymidine, about 0.1 mM, about 0.4 μM and about 0.016 mM and the like, respectively, can be mentioned. For selecting a human-mouse hybridoma, ouabain resistance can be used. Because human cell lines are more susceptible to ouabain than mouse cell lines, it is possible to eliminate unfused human cells by adding ouabain at about 10⁻⁷ to 10⁻³ M to the medium.

In selecting a hybridoma, it is preferable to use feeder cells or culture supernatants of certain cells. As the feeder cells, an allogenic cell species having a lifetime limited so that it dies after helping the emergence of hybridoma, cells capable of producing large amounts of a growth factor useful for the emergence of hybridoma with their proliferation potency reduced by irradiation and the like, and the like are used. For example, as the mouse feeder cells, splenocytes, macrophage, blood, thymocytes and the like can be mentioned; as the human feeder cells, peripheral blood mononuclear cells and the like can be mentioned. As examples of the cell culture supernatant, primary culture supernatants of the above-described various cells and culture supernatants of various established cell lines can be mentioned.

Moreover, a hybridoma can also be selected by reacting a fluorescein-labeled antigen with fusion cells, and thereafter separating the cells that bind to the antigen using a fluorescence-activated cell sorter (FACS). In this case, efforts for cloning can be lessened significantly because a hybridoma that produces an antibody against the target antigen can be directly selected.

For cloning a hybridoma that produces a monoclonal antibody against the target antigen, various methods can be used.

It is preferable to remove aminopterin as soon as possible because it inhibits many cell functions. In the case of mice and rats, aminopterin can be removed 2 weeks after fusion and beyond because most myeloma cells die within 10 to 14 days. However, a human hybridoma is normally maintained in a medium supplemented with aminopterin for about 4 to 6 weeks after fusion. It is desirable that hypoxanthine and thymidine be removed more than one week after the removal of aminopterin. That is, in the case of mouse cells, for example, a complete medium (e.g., RPMI1640 supplemented with 10% FCS) supplemented with hypoxanthine and thymidine (HT) is added or exchanged 7 to 10 days after fusion. About 8 to 14 days after fusion, visible clones emerge. Provided that the diameter of clone has reached about 1 mm, the amount of antibody in the culture supernatant can be measured.

The measurement of the amount of antibody can be performed by, for example, a method comprising adding the hybridoma culture supernatant to a solid phase (e.g., microplate) to which the target antigen or derivatives thereof or partial peptide thereof (including partial peptide used as antigenic determinant) is adsorbed directly or with a carrier, subsequently adding an anti-immunoglobulin (IgG) antibody (an antibody against IgG derived from an animal of the same species as the animal from which the original antibody-producing cells are derived is used) or protein A, which had been labeled with a radioactive substance (e.g., ¹²⁵I, ¹³¹I, ³H, ¹⁴C), enzyme (e.g., β-galactosidase, β-glucosidase, alkaline phosphatase, peroxidase, malate dehydrogenase), fluorescent substance (e.g., fluorescamine, fluorescein isothiocyanate), luminescent substance (e.g., luminol, luminol derivative, luciferin, lucigenin) and the like, and detecting the antibody against the target antigen (antigenic determinant) bound to the solid phase, a method comprising adding the hybridoma culture supernatant to a solid phase to which an anti-IgG antibody or protein A is adsorbed, adding the target antigen or derivatives thereof or partial peptide thereof labeled with the same labeling reagent as described above, and detecting the antibody against the target antigen bound to the solid phase and the like.

Although limiting dilution is normally used as the cloning method, cloning using soft agar and cloning using FACS (described above) are also possible. Cloning by limiting dilution can be performed by, for example, the following procedures, which, however, are not to be construed as limiting.

The amount of antibody is measured as described above, and positive wells are selected. Selected suitable feeder cells are previously added to a 96-well plate. Cells are collected from the antibody-positive wells and suspended in complete medium (e.g., RMPI1640 supplemented with 10% FCS and P/S) to obtain a density of 30 cells/mL; 0.1 mL (3 cells/well) of this suspension is added to the well plate with feeder cells added thereto; a portion of the remaining cell suspension is diluted to 10 cells/mL and sown to other wells (1 cell/well)in the same way; the still remaining cell suspension is diluted to 3 cells/mL and sown to other wells (0.3 cells/well). The cells are cultured for about 2 to 3 weeks until a visible clone appears, when the amount of antibody is measured to select positive wells, and the selected cells are recloned. In the case of human cells, cloning is relatively difficult, so that a plate in which cells are seeded at 10 cells/well is also prepared. Although a monoclonal antibody-producing hybridoma can be obtained normally by two times of subcloning, it is desirable to repeat recloning regularly for several more months to confirm the stability thereof.

### (b) Differential Screening

The hybridomas producing a monoclonal antibody against iPS or ES cells obtained as described above are then subjected to the second screening. In the second screening, not only iPS or ES cells used as immunogen, but also pluripotent stem cells such as ES or iPS cell, EC cell, EG cell, mGS cell and the like can also be used as the probe. As a result of the second screening, a hybridoma producing a monoclonal antibody that reacted with iPS and ES cells but not with pluripotent stem cells other than iPS and ES cells such as ES cells, and somatic cells can be selected as a hybridoma producing an anti-iPS/ES cell antibody of the present invention.

Hybridomas thus obtained can be cultured in vitro or in vivo. As a method of in vitro culture, a method comprising gradually scaling up a monoclonal antibody-producing hybridoma obtained as described above, from a well plate, while keeping the cell density at, for example, about 10⁵ to 10⁶ cells/mL, and gradually lowering the FCS concentration, can be mentioned. As a method of in vivo culture, for example, a method comprising an intraperitoneal injection of a mineral oil to a mouse (a mouse that is histocompatible with the parent strain of the hybridoma) to induce plasmacytoma (MOPC) 5 to 10 days later, to which intraperitoneally injecting about 10⁶ to 10⁷ cells of hybridoma, and collecting ascites fluid under anesthesia 2 to 5 weeks later, can be mentioned.

### (c) Purification of Monoclonal Antibody

Separation and purification of the monoclonal antibody are performed according to a method known per se, for example, a method of immunoglobulin separation and purification [e.g., salting-out, alcohol precipitation, isoelectric point precipitation, electrophoresis, adsorption-desorption with an ion exchanger (e.g., DEAE, QEAE), ultracentrifugation, gel filtration, specific purification comprising selectively collecting the antibody alone by means of an antigen-bound solid phase or an active adsorbent such as protein A or protein G, and dissociating the binding to obtain the antibody, and the like].

As described above, a monoclonal antibody can be produced by culturing a hybridoma in or outside the living body of a warm-blooded animal, and harvesting the antibody from the body fluid or culture thereof.

Examples of the anti-iPS/ES cell antibody of the present invention obtained as mentioned above include mouse anti-human iPS/ES cell antibody mAb R-17F described in the below-mentioned Examples. Hybridoma (R-17F) producing this antibody was deposited on Oct. 11, 2012 at incorporated administrative agency, International Patent Organism Depositary of National Institute of Technology and Evaluation (2-5-8, Kazusakamatari, Kisarazu-shi, Chiba, Japan) under accession No. NITE P-1425, and transferred to an international deposit based on Budapest Treaty on Oct. 8, 2013 under accession No. NITE BP-01425.

### (d) Production of Recombinant Antibody

In another embodiment, cDNAs that encode the heavy chain and light chain of an anti-iPS/ES cell antibody thus obtained can be isolated from cDNA library derived from a hybridoma producing the antibody and cloned into appropriate expression vector(s) functional in a host cell of interest by conventional methods. Then, a host cell is introduced with the heavy chain and light chain expression vector(s) thus obtained. Useful host cells include animal cells, for example, mouse myeloma cells as described above, as well as Chinese hamster ovary (CHO) cells, monkey-derived COS-7 cells, Vero cells, rat-derived GHS cells and the like. Although this introduction can be achieved by any method that is applicable to animal cells, it is preferable to use electroporation or a method based on a cationic lipid and the like. After the host cell is cultured in a suitable medium for a given period, the conditioned medium is recovered, and the antibody protein is purified by a conventional method, whereby the antibody of the present invention can be isolated. Alternatively, by producing a transgenic animal by a conventional method using a germline cell of an animal as a host cell for which transgenic technology has been established, and for which know-how for mass propagation for a domestic animal (poultry) has been compiled, such as bovine, goat or chicken, it is also possible to obtain a large amount of the antibody of the present invention easily from the milk or egg of the animal thus obtained. Furthermore, it is also possible to obtain a large amount of the antibody of the present invention from seeds, leaves and the like obtained from a transgenic plant prepared by microinjection or electroporation for protoplast, the particle gun method, the Ti vector method and the like for intact cells, using as the host cell a cell of a plant for which transgenic technology has been established, and which is cultured in large amounts as a major crop, such as corn, rice, wheat, soybean or tobacco.

When the antibody of the present invention is used to eliminate undifferentiated iPS/ES cells remaining in a differentiated cell population induced from iPS/ES cells, iPS/ES cells are killed by contacting the differentiated cell population and said antibody in vitro, said antibody is removed from the surviving cells, and the differentiated cell population is utilized for cell transplantation and the like. Therefore, the antibody of the present invention does not always need to be humanized. However, the antibody of the present invention can also be a chimeric antibody or a humanized antibody suitable for administration to human, since the risk of post-transplantation tumor formation by undifferentiated iPS/ES cells possibly remaining in the differentiated cell population induced from iPS/ES cells can be reduced by administering the antibody of the present invention along with transplantation of the differentiated cell population to human.

### (e) Production of Chimeric Antibody

In the present specification, the “chimeric antibody” means an antibody wherein the sequences of the variable regions (V_{H} and V_{L}) of the heavy chain and light chain are derived from non-human animal species, and the sequences of the constant regions (C_{H} and C_{L}) are derived from human. The sequence of variable region is preferably derived from animal species capable of easily producing hybridoma, such as mouse, rat, rabbit and the like, and the sequence of constant region is preferably derived from animal species to be the administration subject.

Examples of the production method of the chimeric antibody include the method described in U.S. Pat. No. 6,331,415, such method with partial modification and the like.

A host cell is transformed with the obtained chimeric heavy chain and chimeric light chain expression vectors. As the host cell, transformation method and the like, those exemplified in the production of the above-mentioned (d) recombinant antibody can be preferably used similarly.

### (f) Humanized Antibody

In the present specification, “humanized antibody” means an antibody wherein the sequences of all regions except the complementarity determining region (CDR) present in the variable region (i.e., framework region (FR) in constant region and variable region) are derived from human, and the sequence of CDR alone is derived from other mammal species. As other mammal species, those capable of easily producing hybridoma, such as mouse, rat, rabbit and the like are preferable.

Examples of the production method of the humanized antibody include the methods described in U.S. Pat. Nos. 5,225,539, 5,585,089, 5,693,761, 5,693,762, EP-A-239400, and WO 92/19759, such methods with partial modification, and the like. To be specific, in the same manner as in the above-mentioned chimeric antibody, DNA encoding V_{H} and V_{L} derived from mammal species (e.g., mouse) other than human is isolated, sequencing is performed using an automatic DNA sequencer (e.g., manufactured by Applied Biosystems etc.) according to a conventional method, the obtained base sequence or amino acid sequence deduced therefrom is analyzed using known antibody sequence databases [for example, Kabat database (Kabat et al., “Sequences of Proteins of Immunological Interest”, US Department of Health and Human Services, Public Health Service, ed. NIH, 5th printing, 1991) etc.], and CDR and FR of the both chains are determined. A base sequence is designed by substituting the CDR coding region of a base sequence encoding the light chain and heavy chain of a human antibody having an FR sequence similar to the determined FR sequence, with a base sequence encoding the determined heterologous CDR, the base sequence is divided into fragments of about 20-40 bases, and a sequence complementary to the base sequence is further divided into fragments of about 20-40 bases to permit alternate overlapping with the aforementioned fragments. Each fragment is synthesized by a DNA synthesizer, and hybridized and ligated according to a conventional method, whereby DNA encoding V_{H} and V_{L} having FR derived from human and CDR derived from other mammal species can be constructed. To more rapidly and efficiently transplant CDR derived from other mammal species into V_{H} and V_{L} derived from human, induction of site specific mutation by PCR is preferably used. Examples of such method include the sequential CDR transplantation method described in JP-A-5-227970 and the like.

In the production of humanized antibody by the above-mentioned method, when only the amino acid sequence of CDR is transplanted into human antibody FR as a template, the antigen binding activity may decrease from that of the original non-humanized antibody. In this case, concurrent transplantation of some FR amino acids surrounding CDR is effective. Examples of the non-human antibody FR amino acid to be transplanted include an amino acid residue important for maintaining the steric structure of each CDR, and such amino acid residue can to be assumed by steric structure prediction using a computer.

By linking the thus-obtained DNA encoding V_{H} and V_{L} with a DNA encoding C_{H} and C_{L} derived from human and introducing same into a suitable host cell, a cell or transgenic animal or plant producing the humanized antibody can be obtained.

Examples of the alternative method for producing a humanized antibody without using CDR-grafting for transplanting mouse CDR into a variable region of a human antibody include a method for determining which amino acid residue in non-human variable region is a candidate for substitution, based on the structure-function correlation conserved between antibodies. This method can be performed according to the descriptions of, for example, EP-B-0571613, U.S. Pat. No. 5,766,886, U.S. Pat. No. 5,770,196, U.S. Pat. No. 5,821,123, U.S. Pat. No. 5,869,619 and the like. Using the method, humanized antibody can be easily produced by utilizing, for example, entrusted antibody production service provided by Xoma Corporation, once each amino acid sequence information of V_{H} and V_{L} of the original non-human antibody is obtained.

Humanized antibody can be altered into scFv, scFv-Fc, minibody, dsFv, Fv and the like by using a genetic engineering method, like chimeric antibody, and can also be produced by microorganisms such as Escherichia coli, yeast and the like by using a suitable promoter.

### [III] Use of the Antibody of the Present Invention

Since the antibody of the present invention is capable of specifically recognizing iPS and ES cells, it can be used for detection and quantitation of iPS cells or ES cells in a test cell sample, particularly for detection and quantitation by immunocytochemistry. For these purposes, the antibody molecule itself may be used, and any fragment thereof, such as the F(ab′)₂, Fab′ or Fab fraction of the antibody molecule, may also be used. The measurement method using an antibody against iPS/ES cells should not be particularly limited, and any measurement method can be used.

As the labeling agent to be used for the measurement method using a labeling substance, for example, a radioisotope, an enzyme, a fluorescent substance, a luminescent substance and the like can be used. As the radioisotope, for example, [¹²⁵I], [¹³¹I], [³H], [¹⁴C] and the like can be used. The above-described enzyme is preferably stable and has a high specific activity and, for example, β-galactosidase, β-glucosidase, alkaline phosphatase, peroxidase, malate dehydrogenase and the like can be used. As the fluorescent substance, for example, fluorescamine, fluorescein isothiocyanate (FITC), phycoerythrin (PE) and the like can be used. As the luminescent substance, for example, luminol, luminol derivative, luciferin, lucigenin and the like can be used.

The antibody of the present invention may be directly or indirectly labeled with a labeling agent. In a preferable embodiment, the anti-iPS/ES cell antibody is an unlabeled antibody and iPS/ES cells can be detected by the labeled second antibody such as anti-serum or anti-Ig antibody against the animal from which the anti-iPS/ES cell antibody was produced. Alternatively, the biotinylated second antibody can be used and a conjugate of iPS or ES cell-the antibody of the present invention-the second antibody can be formed and visualized using a labeled streptavidin.

For example, a test cell sample can be fixed and permeabilized with glutaraldehyde, paraformaldehyde or the like, washed with a buffer such as PBS, blocked with BSA or the like and incubated with an anti-iPS/ES cell antibody of the present invention. After washing with a buffer such as PBS to remove unreacted antibody, the cells reacted with the anti-iPS/ES cell antibody can be visualized with the labeled second antibody and analyzed using a confocal laser scanning microscope, a flexible automated cell imaging system IN Cell Analyzer (Amarsham/GE) and the like.

In another embodiment, the antibody of the present invention can be used to isolate (remove) iPS or ES cells from a sample containing the same. Examples of the sample (possibly) containing iPS or ES cells include any differentiated cell population obtained by differentiation induction of iPS or ES cells, a passage culture sample of iPS or ES cells and the like.

For this purpose, for example, the antibody of the present invention may be immobilized on a solid phase comprising any suitable matrix such as agarose, acrylamide, Sepharose, Sephadex and the like. The solid phase may also be any suitable culture vessel such as a microtiter plate. iPS or ES cells in a sample is immobilized on the solid phase when the sample is brought into contact with the solid phase. The cells can be released from the solid phase using an appropriate elution buffer.

In a preferable embodiment, the antibody of the present invention is immobilized on magnetic beads such that iPS or ES cells can be separated from the sample upon provision of a magnetic field (i.e., magnetic activated cell sorting; MACS). In another preferable embodiment, the antibody of the present invention is directly or indirectly labeled with any suitable fluorescent molecule as exemplified above and iPS or ES cells are isolated using a fluorescence activated cell sorter (FACS).

As mentioned above, the anti-iPS/ES cell antibody of the present invention preferably has a cytotoxic activity specific to the target cell. Therefore, when said antibody is used, a separation operation as mentioned above is not necessary, and a uniform differentiated cell population free of undifferentiated cell can be obtained by simply incubating a cell sample for a given time in a medium containing said antibody and harvesting the survived cells, since unnecessary iPS or ES cells present in the sample can be killed or eliminated thereby.

In the case of anti-iPS/ES cell antibody R-17F described in the below-mentioned Examples, the cytotoxic activity against the target cell is remarkably enhanced by adding a trace amount of a secondary antibody to said antibody. Therefore, in a preferable embodiment, a cell sample can be incubated in the presence of the target cytotoxic anti-iPS/ES cell antibody of the present invention and a secondary antibody to said antibody.

The differentiated cells to be provided for the production of the uniform differentiated cell population of the present invention are provided by differentiating iPS or ES cells into desired somatic cells according to a differentiation induction method known per se.

For example, the cells can be differentiated into hematopoietic progenitor cells by coculture with C3H10T1/2 cell line, obtained by exposing human ES cells to radiation irradiation, to induce a saccular structure (ES-sac) (Blood, 111: 5298-306, 2008). As a method for differentiation induction of ES cells into neural stem cell, nerve cell, various methods such as embryoid formation method (Mech Div 59(1) 89-102, 1996), retinoic acid method (Dev Biol 168(2) 342-57, 1995), SDIA method (Neuron 28(1) 31-40, 2000), NSS method (Neurosci Res 46(2) 241-9, 2003) and the like are known. As a method of inducing ES cell into cardiomyocyte, a method of adding factors such as retinoic acid, TGFβ1, FGF, dynorphin B, ascorbic acid, nitric oxide, FGF2 and BMP2, Wnt11, PP2, Wnt3a/Wnt inhibitor and the like to a medium, the cardiac muscle differentiation induction method of Noggin (Nat Biotechnol 23(5) 611, 2005) and the like have been reported heretofore. Furthermore, a differentiation induction method of ES/iPS cells into retinal cells by the SDIA method and SFEB method (Nat Neurosci 8 288-96, 2005) and the like are known, but the method is not limited thereto.

The cell population differentiation-induced from iPS/ES cells obtained as mentioned above and the antibody of the present invention can be contacted by adding a suitable concentration of the antibody of the present invention (and a secondary antibody) to a medium suitable for culture of differentiated cells, and incubating the differentiated cell population for a given time. While the concentration of the antibody of the present invention to be added varies depending on the kind of the antibody, cell density, reaction temperature, reaction time and the like, it can be appropriately selected from the range of, for example, 0.1-1000 μg/mL, preferably 1-100 μg/mL. The reaction temperature is not particularly limited as long as it is suitable for the survival of differentiated cells, and can be appropriately selected from the range of 0-40° C., preferably 20-40° C., more preferably 30-40° C. While the reaction time is not particularly limited as long as it is sufficient to induce cell death of iPS or ES cells and does not adversely influence the survival of differentiated cells, it is, for example, within 3 hr, preferably 1 min-2 hr, more preferably 15 min-1 hr. When the secondary antibody is further added, the concentration thereof is not particularly limited as long as it enhances the cytotoxic activity of the antibody of the present invention and does not show cytotoxicity against the differentiated cells. It can be appropriately selected from the range of, for example, 0.01-10 μg/mL, preferably 0.1-1.0 μg/mL, more preferably 0.2-0.5 μg/mL.

After completion of the reaction, the medium is removed, the cells are washed with a fresh medium or a suitable buffer such as PBS and the like, and viable cells are recovered by a conventional method, whereby a uniform differentiated cell population, wherein undifferentiated cells have been killed and eliminated, can be obtained.

The uniform differentiated cell population obtained as mentioned above is produced as a parenteral preparation for cell transplantation such as injection, suspension, drip infusion and the like, by mixing same with a pharmaceutically acceptable carrier according to a conventional means. Examples of the pharmaceutically acceptable carrier that can be contained in the parenteral preparation include aqueous solutions for injection such as physiological saline, isotonic solution (e.g., D-sorbitol, D-mannitol, sodium chloride and the like) containing glucose, other auxiliary agents and the like. The transplantation therapy agent of the present invention may be mixed with, for example, buffering agent (e.g., phosphate buffer, sodium acetate buffer), soothing agent (e.g., benzalkonium chloride, procaine hydrochloride and the like), stabilizer (e.g., human serum albumin, polyethylene glycol and the like), preservative, antioxidant and the like. When the transplantation therapy agent of the present invention is formulated as an aqueous suspension, the differentiated cells may be suspended in the above-mentioned aqueous solution at about 1×10⁶-about 1×10⁸ cells/mL.

The transplantation therapy agent of the present invention can also be provided in a state of cryopreservation under the conditions generally used for cryopreservation of cells, and used by melting when in use. In this case, it may further contain a serum or an alternative thereof, an organic solvent (e.g., DMSO) and the like. In this case, the concentration of the serum or an alternative thereof is not particularly limited, and can be about 1-about 30% (v/v), preferably about 5-about 20% (v/v). The concentration of the organic solvent is not particularly limited and can be 0-about 50% (v/v), preferably about 5-about 20% (v/v).

As mentioned above, the antibody of the present invention can be administered to patients in need of cell transplantation by combining with a cell population differentiation-induced from iPS/ES cells.

A medicament containing the antibody of the present invention as an active ingredient can be administered after formulation by a known drug formulation method. For example, it can be used in the form of an injection of a sterile solution with water or other pharmaceutically acceptable solution, or a suspension. In addition, it may be formulated by, for example, appropriately combining with a pharmacologically acceptable carrier or medium, concretely, sterile water, physiological saline, emulsifier, suspending agent, surfactant, stabilizer, vehicle, preservative and the like, and admixing in a generally-known unit dosage form requested for practicing drug formulation. The amount of the active ingredient in these preparations is such an amount that affords a suitable volume within the indicated range.

An aseptic composition for injection can be formulated using a vehicle such as distilled water for injection and according to general practice of drug formulation. Examples of the aqueous solution for injection include physiological saline, and isotonic solution containing glucose and other auxiliary drugs, for example, D-sorbitol, D-mannose, D-mannitol, and sodium chloride, and it may be used in combination with a suitable solubilizing agent, for example, alcohol, concretely, ethanol, polyalcohol, such as propylene glycol, polyethylene glycol, non-ionic surfactant, such as polysorbate 80™, and HCO-50.

Examples of an oily liquid include sesame oil and soybean oil, and it may be used as a solubilizing agent in combination with benzyl benzoate and benzyl alcohol. It may also be mixed with a buffering agent such as phosphate buffer, sodium acetate buffer, a soothing agent such as procaine hydrochloride, a stabilizer such as benzyl alcohol, phenol, and an antioxidant. The prepared injection is generally filled in a suitable ampoule.

A medicament containing the antibody of the present invention as an active ingredient can be administered orally or parenterally, and preferred is parenteral administration. Specific examples thereof include injection dosage form, transnasal administration dosage form, pulmonary administration dosage form, transdermal administration dosage form and the like. As an example of the injection dosage form, the medicament can be administered systemically or locally by intravenous injection, intramuscular injection, intraperitoneal injection, subcutaneous injection and the like.

The dose can be appropriately determined according to the age and symptom of patients. For example, a single dose can be determined within the range of 0.0001 mg-1,000 mg/kg body weight. Alternatively, it can also be determined within the range of 0.001-100,000 mg per patient. The administration period can be appropriately determined from before transplantation of cell population differentiation-induced from iPS/ES cells, simultaneously with the transplantation or after the transplantation. The number of administration and administration interval are not particularly limited, and the administration may be performed once, or 2-6 times at, for example, 2-8 weeks intervals.

The present invention is described in further detail below by means of the following Examples, to which, however, the invention is not limited.

### EXAMPLES

### [Materials and Methods]

### 1) Antibodies

Anti-human TRA-1-60 monoclonal antibody (Clone #TRA-1-60, mouse IgM), anti-human TRA-1-81 monoclonal antibody (Clone #TRA-1-81, mouse IgM), anti-human/mouse SSEA-4 monoclonal antibodies (clone #MC813, mouse IgG3) were obtained from Santa Cruz Biotechnology, Inc. (Santa Cruz, Calif.). Anti-human/mouse SSEA-1 antibody (clone #MC480, mouse IgM), anti-human/mouse SSEA-3 monoclonal antibody (clone #MC631, rat IgM) and anti-human podocalyxin monoclonal antibody (clone #222328, mouse IgG_{2A}) were obtained from R & D Systems, Inc. (Minneapolis, Minn.). Anti-human podocalyxin-like protein I (clone mAb 84, mouse IgM) was obtained from Millipore (Billerica, Hercules, Calif.). Anti-human Nanog monoclonal antibody and anti-human Oct4 monoclonal antibody were obtained from ReproCELL (Kanagawa, Japan) and Abcam (Cambridge, UK), respectively.

### 2) Cells and Cell Culture

Human iPS cell lines, Tic (JCRB1331) and human EC cell line NCR-G3 (JCRB1168) was obtained from JCRB, National Institute of Biomedical Innovation (Osaka), 201B2 and 201B7 were provided from the Center for iPS Cell Research and Application (CiRA), Kyoto University. Human ES cell line, KhES-3 was provided from the Institute for Frontier Medical Sciences, Kyoto University and H9 was provided from Wisconsin International Stem Cell Bank, WiCell (Madison, Wis.), respectively. These cells were cultured at 37° C./5% CO₂ on mitomycin C-treated feeders (mouse embryonic fibroblasts (MEF), 5×10³ cells/cm²) in rectangular canted neck cell culture flask with vent cap (25 cm², Corning, N.Y.). Human embryonal carcinoma cell line, 2102Ep, was a generous gift from Prof. Peter Andrews (University of Sheffield). MRC-5 (JCRB9008), fibroblast-like cell line derived from human lung tissue of a 14-week-old (male) fetus was obtained from JCRB cell bank.

3) Production of Human iPS cells for Immunization and for Screening

Human iPS cell line, Tic, which was generated from MRC-5 (Toyoda et al., 2011), human embryonic fibroblasts, by transduction of four reprogramming genes: Oct3/4, Sox2, Klf4, and c-Myc (Takahashi et al., 2007), was used as immunogens and also as the screening probe. Tic cells maintained in a serum free media, ES medium (KNOCKOUT DMEM/F-12 (400 mL, Invitrogen-Life technologies, Carlsbad, Calif.), MEM non-essential amino acids solution (4.0 mL, Invitrogen-Life technologies, Carlsbad, Calif.), 200 mM L-glutamine (5.0 mL), KNOCKOUT Serum Replacement (100 mL, Invitrogen-Life technologies, Carlsbad, Calif.), and 55 mM 2-mercaptoethanol (0.925 mL), 10 μg/mL FGF-Basic human (Sigma) added to 1000-fold dilution (hereinafter iPS culture medium)), were transferred to hESF9 medium, which comprises ESF basal medium (Cell Science and Technology Institute, Sendai, Japan, Furue et al., 2005) without HEPES supplemented with ascorbic acid 2-phosphate ester, 6-factors (human recombinant insulin, human transferrin, 2-mercaptoethanol, 2-ethanolamine, sodium selenite, oleic acid conjugated with fatty acid-free bovine serum albumin (FAF-BSA)), bovine heparan sulfate sodium salt, and human recombinant FGF-2 (Katayama Chemical Industries, Osaka), as described previously (Furue et al., 2008). After incubation at 37° C. for 4-5 days, the cells in one group of flasks (3×10⁵-1×10⁶ cells/25 cm² flask) were harvested by treating with 0.1% EDTA-4Na (1 ml/flask), collected by centrifugation at 1,000 rpm for 2 min, washed with PBS and stored at −80° C. until just before use as immunogens. The cells in other group of flasks were used for the preparation of cell screening plates. To these flasks ROCK inhibitor (10 μM, Y27632, Wako Pure Chemical, Osaka) was added to permit survival of dissociated cells (Watanabe et al., 2007). After incubation at 37° C. for 1 h, the cells were harvested with accutase (1 mL, Millipore, Billerica, Mass.) collected by centrifugation, washed with S-medium, suspended in hESF9 medium, and seeded in fibronectin coated 96-well plates (5×10³ cells/well, BD, Franklin Lakes, N.J.). Cells were fixed with 1% acetic acid/ethanol (100 μl/well) for 15-30 min. After washing with PBS, the plates were stored at −80° C. until just before use.

### 4) Immunization

Two different protocols were used for the immunization of mice with human iPS cells. In protocol A, the freeze-thawed Tic cells (1.5×10⁷ cells in 0.5 mL PBS) were emulsified with an equal volume of Freund's Complete Adjuvant (CFA, Thermo Fisher Scientific, Rockford, Ill.) and injected into 8-week-old female C57BL/6 mice (200 μL/mice)intraperitoneally on day 0. Thereafter, booster injection was performed on day 25, and the mice were sacrificed on day 28. In protocol B, FCA emulsion of Tic cells was injected subcutaneously into mice (200 μL/mice) and the mice were sacrificed after 2 weeks.

### 5) Cell Fusion and Cloning

Lymphocytes from the spleen of the protocol A mice and lymph nodes from the protocol B mice were mixed and fused with P3U1 myeloma cells using polyethylene glycol. Fused cells were seeded in 96-well tissue culture plates, and hybridoma were selected by adding the hybridoma medium (S-Clone cloning medium CM-B containing hypoxanthine, aminopterin and thymidine (HAT), Sanko Junyaku, Tokyo). On the day 7 after plating, the first screening was performed using Tic cell fixed plates. Culture supernatant from each hybridoma was added to Tic cell-fixed screening plates, which had been pretreated with blocking solution containing 0.1% H₂O₂ (Blocker Casein, Pierce-Thermo Fisher Scientific, Rockford, Ill.), overnight. The hybridoma culture supernatant was incubated in the cell plates at room temperature for 2 hour. After washing the plates with PBS, 1:2000 diluted horseradish peroxidase (HRP) conjugated anti mouse IgG (Takara-bio, Shiga) was added in each well and incubated for 1 hour. After washing, chromogenic substance DAB (Metal Enhanced DAB Substrate Kit, Pierce-Thermo Fisher Scientific, Rockford, Ill.) was added to the plates and colored for 10-15 min, followed by observing the stained plates under the light microscope (Olympus IX 7, Olympus, Tokyo).

The human iPS cell positive antibody producing hybridomas were then subjected to the second cell screening. In the second screening, human EC cells (2102Ep), original human fibroblasts (MRC-5) and mouse embryonic fibroblast (MEF) cells were used as probes besides human iPS cells (Tic). Isotype of monoclonal antibody was analyzed by using mouse monoclonal antibody isotyping test kit (AbD Serotec, Kidlington, UK).

### 6) Immunocytochemistry

Cells seeded in 24-well plates were fixed in 4% paraformaldehyde (PFA) at room temperature for 15 min, blocked with 3% FBS/PBS for 1 hour, and then incubated with various monoclonal antibodies (R-10G, TRA-1-60, TRA-1-81, SSEA-4, SSEA-3, SSEA-1, mAb84, Nanog, Oct4 and anti-PODXL antibody) at 4° C. overnight. After washing with 0.1% FBS/PBS three times (each for 5 min), localization of antibodies was visualized by incubation with Alexa Fluor 647-labeled chicken anti-mouse IgG antibody (Invitrogen-Life technologies, Carlsbad, Calif.) as the second antibody at room temperature for 1 hour, followed by staining with Hoechst 33342 (1:5000 in PBS, Dojindo Laboratories, Kumamoto). Then the cells were analyzed using In Cell analyzer 2000 (GE Healthcare, Buckinghamshire, UK) and Developer Toolbox ver 1.8.

### 7) Laser Confocal Scanning Microscope

Tic cells were seeded to Millipore EZ slides (Millipore, Billerica, Mass.), which had been coated with gelatin, and plated with B6 mouse-derived MEF. After two day's culture, cells were fixed in 4% PFA at room temperature for 10 minutes, blocked with 3% BSA/PBS for 1 hour and then incubated with monoclonal antibody R-17F (as a first primary antibody) at 4° C. overnight. After washing with 0.1% BSA/PBS three times, cells were incubated with Alexa Fluor 488-labeled goat anti-mouse IgG1 antibody (as the secondary antibody) in 1% BSA/PBS at room temperature for 30-60 min.

For double (and triple) staining with the first to third primary antibodies, cells were washed and blocked in the same way as described above and incubated with the first to third primary antibodies (R-17F, SSEA-3 and SSEA-4) at 4° C. overnight. Then, the cells were incubated with Alexa Fluor 488-labeled goat anti-mouse IgG1 antibody (secondary antibody to R-17F), Alexa Fluor 594-labeled rat anti-mouse IgM antibody (secondary antibody to SSEA-3) and Alexa Fluor 594-labeled goat anti-mouse IgG3 antibody (secondary antibody to SSEA-4) in the same manner as above. After washing with 0.1% BSA/PBS three times, the cells were fixed with 0.1% Triton X-100/4% PFA at room temperature for 10 min, followed by staining with TO-PROS (500-fold diluted with PBS, Invitrogen-Life technologies, Carlsbad, Calif.) and monitored using confocal laser scanning microscope FV1000 (Olympus, Tokyo).

### 8) Purification of Monoclonal Antibody R-17F From Mouse Ascites Fluid

R-17F hybridoma cell line was injected intraperitoneally into pristane-treated SCID mice (CB-17/Icr-scid Jcl). Two weeks later, the ascites fluid (2.5 mL) were collected from the mice and subjected to a Protein A-Sepharose column (1×6.0 cm) (GE Healthcare, Buckinghamshire, UK). Monoclonal antibody R-17F bound to the column in 1.5 M Glycine-NaOH buffer, (pH 8.9)/3M NaCl and was eluted with 0.1 M citric acid-phosphate buffer (pH 4.0). The eluate containing monoclonal antibody R-17F was immediately neutralized to pH 7-8 by adding 3M Tris-HCl buffer (pH 9.0).

### 9) SDS-PAGE and Western Blotting

SDS-PAGE and Western blotting were performed according to the methods of Laemmli (1970) and Towbin et al. (1992), respectively. Briefly, samples were resolved by electrophoresis on a 4-15% gradient SDS-polyacrylamide gel (Mini-PROTEAN TGX-gel, BioRad Laboratories, Hercules, Calif.) under non-reducing conditions and followed by either Western blotting or protein staining. For Western blotting, resolved proteins were transferred onto Immobilon Transfer membrane (Millipore, Billerica, Mass.), followed by immunoblot detection with specific antibody. For visualization, a chemiluminescent substrate kit (Pierce-Thermo Scientific, Rockford, Ill.) was used with HRP-labeled rabbit anti-mouse immunoglobulins (DAKO Cytomation, Denmark A/S), followed by analysis with Luminolmage Analyzer, Las 4000 mini (GE Healthcare, Buckinghamshire, UK). Protein was stained by Coomassie brilliant Blue G-250, (GelCode Blue, Invitrogen-Life technologies, Carlsbad, Calif.).

### 10) Flow Cytometry

### Cell Preparation:

Culture medium was removed from a culture flask of human iPS cell line Tic, Dispase (1 mg/mL) (1-2 mL) was added, and the mixture was incubated at 37° C. for about 2 min. After confirmation of curling of the periphery of colony with a microscope, Dispase was removed. A medium for washing (KO-DM/F12 after expiration date) was added, and the cells were scraped with a cell scraper. The obtained cell suspension was centrifuged at 20° C., 300 rpm for 2 min, and the supernatant was removed. Then, PBS (10 mL) was added, the mixture was centrifuged again at 20° C., 300 rpm for 2 min, and the supernatant was removed. To the precipitate was added 0.25% Trypsin/EDTA (500 μL), and the mixture was incubated at 37° C. After 15 min, the mixture was taken out from the incubator and a single cell suspension was obtained by pipetting. FACS buffer (1% BSA/Dulbecco's Phosphate-Buffered Saline (D-PBS) solution, 4° C.) (9.5 mL) was added. Then the mixture was centrifuged at 4° C., 1500 rpm for 3 min, and the supernatant was removed. The precipitate was lightly tapped with a finger, suspended in FACS buffer (0.5-1.0 mL), and the number of the cells was counted (Trypan Blue staining). All subsequent operations were performed at 4° C. or in ice.

### Immunofluorescent Staining:

1×10⁵ Cells/sample were transferred into a 1.5 mL tube, and centrifuged at 4° C., 6000 rpm for 3 min, and the supernatant was removed. To the precipitate were added FACS buffer (1% BSA, 0.1% NaN₃-containing PBS) (100 μL) and then a primary antibody (5 βL) (used by diluting 100-fold to 1000-fold depending on antibody)) and the resulting suspension was left standing in ice for 30-45 min. After the reaction, FACS buffer (1 mL) was added, and the mixture was centrifuged at 4° C., 6000 rpm for 3 min, and the supernatant was removed. This washing operation was repeated twice. Then, to the precipitate were added FACS buffer (100 μL) and a secondary antibody (5 μL) (used by diluting about 100-fold) to give a suspension. All subsequent operations were performed under shading. The suspension was left standing for 30 min in ice to allow for reaction, FACS buffer (1 mL) was added, the mixture was centrifuged at 4° C., 6000 rpm for 3 min, and the supernatant was removed. Similar washing was repeated twice, and the cells were suspended in FACS buffer (1 mL). The suspension was transferred into an FACS tube equipped with a cell strainer, and analyzed by FACS.

### 11) Measurement of Cytotoxic Activity

1×10⁵ Cells/sample were transferred into a 1.5 mL tube and centrifuged at 4° C., 6000 rpm for 3 min, and the supernatant was removed. To the precipitate was added FACS buffer (100 μL), a primary antibody (5 μL) (used by diluting 100-fold to 1000-fold depending on antibody) was added, and the mixture was reacted in ice for 30-45 min. After the reaction, FACS buffer (1 mL) was added, the mixture was centrifuged at 4° C., 6000 rpm for 3 min, and the supernatant was removed. This washing operation was repeated twice, and to the precipitate was added FACS buffer (100 μL). Then, 7-AAD (7-amino-actinomycin D, eBioscience, Inc. San Diego, Calif.) (5 μL, 0.25 μg) was added to give a suspension. All subsequent operations were performed under shading. The sample was transferred into an FACS tube equipped with a cell strainer, left standing at ambient temperature for 5 min, and analyzed by FACS.

### 12) Influence of Glycolipid Synthesis Inhibitor on Expression of R-17F Antibody Epitope

To a medium of Tic cells on day 4 of passage was added D-PDMP(D-threo-1-phenyl-2-decanoylamino-3-morpholino-1-propanol) (Sigma-Aldrich, St. Louis, Mo.) (20 μM), which is a specific inhibitor of sphingoglycolipid glucosyl ceramide (GlcCer) biosynthase, and the mixture was cultured for 4 days. The culture medium was removed, Dispase (1 mg/mL) (1-2 mL) was added, and the mixture was incubated at 37° C. for about 2 min. According to the method mentioned above in 10) Flow cytometry, a single cell suspension was prepared by 0.25% Trypsin/EDTA treatment, and the cells were reacted with SSEA-4, TRA-1-60 and R-17F in ice water for 45 min, transferred into an FACS tube equipped with a cell strainer, and analyzed by FACS.

### 13) Cell Proliferation Suppressive Action of R-17F Antibody on Cultured iPS Cell Colony

Human iPS cells (Tic) were seeded on a chamber slide glass, and cultured in an iPS culture medium for 2 days. Thereafter, to hESF9 medium was added R-10G antibody (100 βg), R-17F antibody (100 μg), or PBS as a control to give 200 μL each. The medium was exchanged, and the cells were cultured for 72 hr. The cells were observed and photographed with a phase contrast microscope at 0 h, 24 h, 48 h and 72 h.

### 14) Extraction of Tic Cellular Lipid and TLC-Immunostaining

To cryopreserved Tic cells (3.0×10⁷ cells) was added 3 mL of chloroform/methanol (2:1, v/v), and the mixture was sonicated at 37° C. for 5 min and extracted at 37° C. for 1 hr. The mixture was centrifuged at 4° C., 2500 rpm for 10 min, and the obtained supernatant was transferred into a glass tube. To the precipitate was added 2 ml of chloroform/methanol/water (1:2:0.8, v/v/v), and the mixture was extracted at 37° C. for 2 hr. The suspension was centrifuged at 4° C., 2500 rpm for 10 min, and the obtained supernatant and the earlier supernatant were combined to give a total lipid extract. The total lipid extract was dissolved in 250 μL of chloroform/methanol/water (65:25:4, v/v/v) to give a TLC analysis sample. HPTLC silica gel 60 alumina plate (Merk) (10 cm×10 cm) was used for TLC. The sample was spotted using Linomat 5 (CAMAG, Muttenz, Switzerland) (5-20 μL), and developed using chloroform/methanol/water (65:25:4, v/v/v) as a solvent. After completion of the development, a primulin reagent (0.001% acetone/water (1:10, v/v) solution) was sprayed on an air-dried TLC plate, and observed at 365 nm by using a UV vertical imaging apparatus (ATTO, Tokyo), and separation of the lipid component was observed. Then, the lipid component separated on the TLC plate was transcribed onto a PVDF membrane according to the method of Taki (Taki&Ishikawa, 1997), by using a TLC heat transcription apparatus (ATTO, AC-5970). That is, the TLC plate was immersed in a blotting solvent (isopropanol/0.20 CaCl₂/methanol (40:20:7, v/v/v)) for 15 sec, and the PVDF membrane, Teflon (registered trade mark) membrane, and glass fiber filter paper were laminated, and transcribed for 30 sec using a heat transcription apparatus heated to 180° C. The transcribed PVDF membrane was blocked in 3% BSA/PBS at 4° C. overnight, and reacted by incubating with R-17F antibody (1 μg/mL) at room temperature for 1.5 hr. Then, it was reacted with biotin-labeled anti-mouse IgG (H+L) (0.1 μg/mL) (Kirkegaard & Perry Laboratories, Inc., MD, USA)) at room temperature for 1 hr, reacted with HRP-labeled streptavidin (55 ng/mL) (Pierce-Thermo Scientific, Rockford, Ill.) for 1 hr, treated with a chemical luminescence reagent (Pierce West Pico, Pierce-Thermo Scientific) for 5 min, and observed by LAS 4000 mini (GE Healthcare, Buckinghamshire, UK).

### 15) Purification of R-17F Antibody Epitope by Preparative TLC

To a center portion (66 mm) of an HPTLC plate (10 cm×10 cm) (HPTLC silica gel 60 F254 MS-grade glass plate, Merck) was applied Tic total lipid (corresponding to 4.0×10⁷ cells) dissolved in 180 μL of chloroform/methanol/water (65:25:4, v/v/v). The HPTLC plate was dried, and developed by 6 cm in a developing chamber of chloroform/methanol/Milli-Q water (65:25:4, v/v/v) (first time of development). The HPTLC plate was air-dried by a dryer and left standing for 10 min. The developed HPTLC plate was developed by 8.5 cm in a developing chamber exchanged with an eluent having the same mixing ratio (second time of development). After development, the HPTLC plate was air-dried, and the operation of the second time of development was repeated, thus performing three times of development in total. The both ends (about 2 cm) of the HPTLC plate after development were cut by a glass cutter (glass cutter 2A with diamond in tip, TOSHIN RIKO CO., LTD.), and the both end portions of the HPTLC plate cut in 10 cm×2 cm were TLC-Immunostained with R-17F (1 μg/mL) and R-17F-bound lipid was detected.

Silica gel of a band portion corresponding to the mobility of R-17F-bound lipid detected by TLC-Immunostaining was scraped from the plate for preparative TLC. The scraped silica gel was transferred into a screw cap glass test tube, and 3 mL of chloroform/methanol/Milli-Q water (65:25:4, v/v/v) was added. The mixture was sonicated from the outside at room temperature in a hot-water bath for 3 min, and left standing at 4° C. overnight to extract the lipid. A glass SPE filter paper (GL Sciences) was set on a glass SPE cartridge (GL Sciences), and the silica gel suspension was filtered by adding thereto. The filtrate (lipid extract) was collected in a spitz type screw cap glass test tube (IWAKI). The glass SPE cartridge after filtration was washed three times with chloroform/methanol/Milli-Q water (65:25:4, v/v/v) (500 μL) and twice with methanol (500 μL). These washings were combined with the filtrate (lipid extract), and dried under a nitrogen gas stream to give an R-17F antibody-bound lipid. The R-17F-bound lipid was dissolved in 150 μL of chloroform/methanol/Milli-Q water (65:25:4, v/v/v), and preserved at 4° C.

### 16) Analysis of Epitope Structure by Mass Spectrometry Apparatus

A sample solution (1-2 μL) was sucked with a glass capillary and applied to a MALDI plate. Thereonto was layered a matrix solution (DHB, 2,5-dihyroxybenzoic acid, 5 mg/mL) and dried. Using SHIMADZU Corporation/Kratos Matrix Assisted Laser Desorption Ionization/Quadrupole Ion Trap/Time of. Flight Mass Spectrometer, AXIMA Resonance (Shimadzu Corporation), the sample was measured in positive mode. The mass spectrum obtained for the sample showed a group of signals assignable to m/z 1000-2000 region. The main peak was measured for MS/MS, and further MS³, and an assumed structure was submitted (this study was performed by cooperation of Mr. Tsuyoshi Okumura and Mr. Shuichi Nakaie of Shimadzu Corporation, Kyoto, Japan).

### 17) Analysis of Binding Specificity of R-17F Antibody by Sugar Chain Microarray

Various purified neoglycolipid sugar chains were applied to NC membrane (Trans-Blot Transfer Medium Pure Nitrocellulose 0.45 μm, Bio-Rad Laboratories, Inc.) (each 2 mm width, 1 pmol, 5 mol), dried, immersed in 3% BSA/PBS, and blocked at 4° C. overnight. After blocking, the NC membrane was transferred into a moisturizing box, R-17F (1 μg/mL) diluted with 1% BSA/PBS was overlaid at 40 mL per 1 cm², and reaction was performed at room temperature for 2 hr.

After the primary antibody reaction, the membrane was washed three times with PBS for 3 min, transferred into another moisturizing box, a secondary antibody (1.3 μg/mL) Rabbit polyclonal anti-mouse Ig-HRP [DAKO] diluted with 1% BSA/PBS was overlaid at 40 μL per 1 cm², and reaction was performed at room temperature for 1 hr. After the reaction, the membrane was washed three times with PBS for 3 min, reacted with a chemical luminescence reagent, SuperSignal West Pico Chemiluminescent Substrate, (Thermo Fisher Scientific, Rockford) for 5 min, and detected in Chemiluminescence mode of Luminescent image analyzer (Las4000miniEPUV, GE Healthcare].

The structures of the sugar chains are shown below. All were ADHP-derivatized and used.
LNFP I: Lacto-N-fucopentaose I
Fuc(a1-2)Gal(β1-3)GlcNAc(β1-3)Gal(β1-4)Glc
LNnT: Lacto-N-neotetraose
Gal(β1-4)GlcNAc(β1-3)Gal(β1-4)Glc
LNT: Lacto-N-tetraose
Gal(β1-3)GlcNAc(β1-3)Gal(β1-4)Glc
Lewis b: Lacto-N-difucohexose I, LNDFH I
Fuc(α1-2)Gal(β1-3)[Fuc(α1-4)]GlcNAc(β1-3)Gal(β1-4)Glc
Lewis a: Lacto-N-fucopentaose II, LNFP II
Gal(β1-3)[Fuc(α1-4)]GlcNAc(β1-3)Gal(β1-4)Glc

### 18) Determination of Base Sequence of Variable Region of R-17F Antibody Gene

Total RNA was purified from hybridoma cell R-17F by using MACHEREY-NAGEL NucleoSpin RNA kit (MACHEREY-NAGEL GmbH & Co. KG, Duren, Germany). Using SMARTer™RACE cDNA Amplification Kit (Clonetech), 5′RACE analysis was performed. Then, H chain cDNA was synthesized by RT reaction using total RNA as a template and mouse antibody (IgG) H chain specific primer (H-RT1). Similarly, L chain cDNA was synthesized using (IgG) L chain specific primer (L-RT1). Using these cDNAs as templates, RACE PCR was performed using mouse antibody (IgG) H chain constant region specific primer (H-PCR) as a reverse primer, and UPM (Universal primer mix) contained in the kit as a forward primer. Similarly, using L chain constant region specific primer (L-PCR) as a reverse primer, RACE PCR was performed. The obtained PCR product was analyzed by agarose gel electrophoresis. PCR products having an assumed size were obtained, they were named as SYN4553H and SYN5531L. PCR products after gel purification were ligated to cloning plasmid pMD20-T. Transformation was performed according to a conventional method, and 48 clones were obtained for each derivation from the PCR products. These clones were analyzed from one side of the plasmid region. For sequence reaction, BigDye Terminators v3.1 Cycle Sequencing Kit (ABI) was used and analysis was performed by ABI3730 Sequencer (ABI). The homology of the obtained base sequences was determined by DNA Sequence Assembling Software, SEQUENCHER™.

The base sequences (5′→3′) of the primers used in the experiment are shown.
RT reaction

H-RT1:
(SEQ ID NO: 11)
TCCAKAGTTCCA
L-RT1:
(SEQ ID NO: 12)
GCTGTCCTGATC

PCR reaction (Reverse Primer)

H-PCR:
(SEQ ID NO: 13)
GGGAARTARCCCTTGACCAGGCA
(SEQ ID NO: 14)
GGGAARTARCCCTTGACCAGGCA

Equimolar amounts of these two sequences were mixed and used.

L-PCR:
(SEQ ID NO: 15)
CACTGCCATCAATCTTCCACTTGACA

### [Results]

### 1. Production of Monoclonal Antibody R-17F Specific to Human iPS Cell

In order to produce a panel of monoclonal antibodies to cell surface markers on human iPS cells, freeze-thawed Tic cells in PBS were mixed with FCA and used to immunize C57BL/6 mice intraperitoneally or subcutaneously. Primary screening of a total of 960 hybridomas using Tic cell fixed plates and MRC-5 fixed plates (control) indicated that 29 clones produced monoclonal antibodies that had reactivity to surface antigens on Tic cells. Secondary screening was performed for these 29 clones to determine the cross reactivity of the monoclonal antibodies with human EC cells such as 2102Ep and mouse feeders (MEF). There was essentially no antibody reactivity with the mouse feeders that human iPS were cultured on prior to immunization. In contrast, many of the monoclonal antibody panel had reactivity with 2102 Ep, an EC cell line. Interestingly, however, monoclonal antibody Nos. 10, 11 and 17 had no or weak reactivity with 2102Ep, indicating clearly that there are differences in surface antigen expression between human iPS and human EC cells.

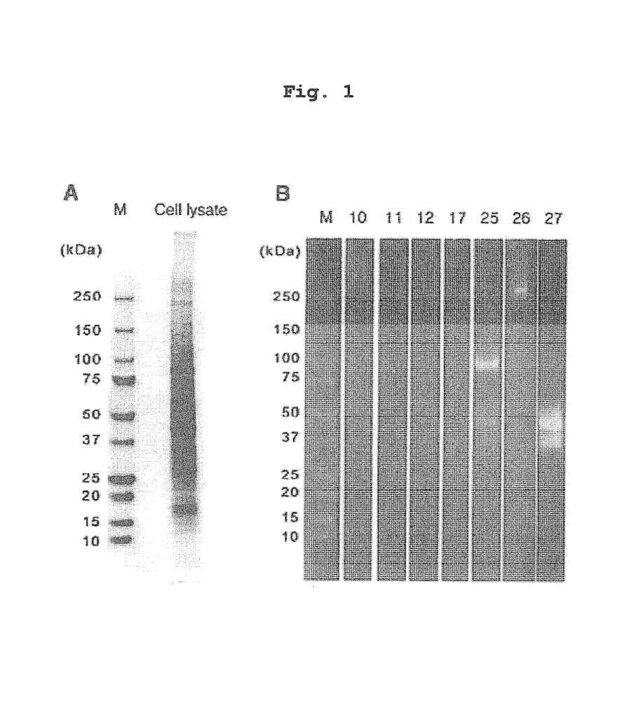

The binding of the monoclonal antibodies to human iPS cells was confirmed by Western blotting, in which Tic cell lysates were resolved by SDS-PAGE (FIG. 1A) and the culture supernatant of hybridomas were tested as primary antibodies. Some of the representative profiles of Western blotting are shown in FIG. 1B. Some monoclonal antibodies showed strong bindability with human iPS cell in cell plate assay, but substantial binding was not detected, or only a slight band could be detected in Western blotting (No. 11, No. 12, No. 17). Therefore, these monoclonal antibodies are considered to react with a cellular surface component other than protein. Among these antibodies, the present inventors focused on the antibody of clone No. 17 (designated as R-17F), which belongs to IgG1 subclass.

### 2. Cell Binding Property of R-17F Antibody

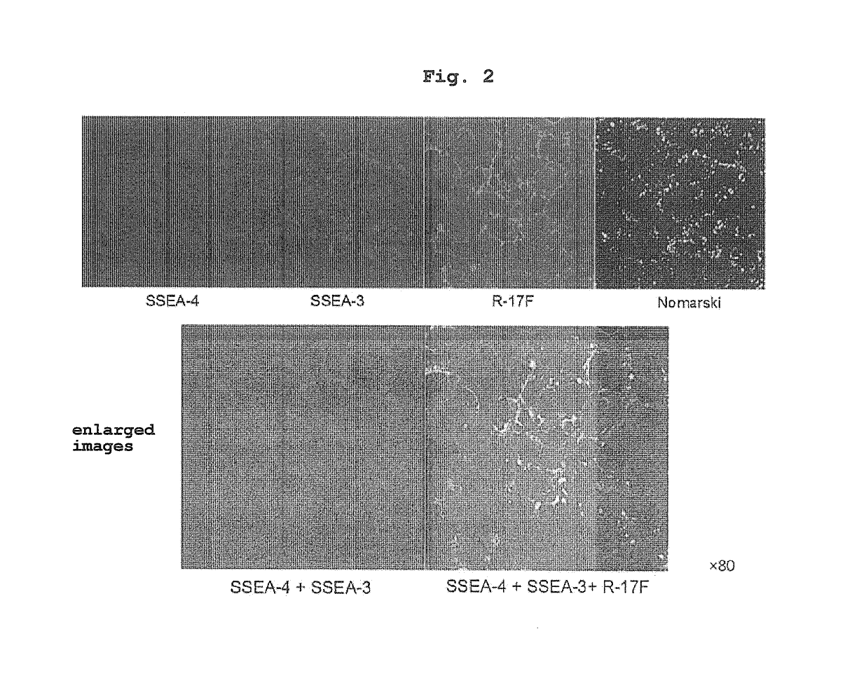

The reactivity of monoclonal antibody R-17F on human iPS cells, Tic, was compared with those of known human iPS/ES cell marker antibodies, TRA-1-60, TRA-1-81, SSEA-4, SSEA-3, SSEA-1, Nanog and Oct-4 and also with those of mAB84, a mouse monoclonal antibody produced against human ES cell line HES-3 (Choo et al., 2008) and anti-podocalyxin antibody against recombinant human podocalyxin. The results are shown in Table 1 and FIG. 2.

TABLE 1
binding property of single clone antibody R-17F to human iPS, ES, EC cells

| | R-17F | R-10G | TRA-1-60 | TRA-1-81 | SSEA-4 | SSEA-3 | SSEA-1 | mAb84 | Nanog | Oct4 | aPODXL |
|---|---|---|---|---|---|---|---|---|---|---|---|
| Tic | ++++ | +++ | ++++ | ++++ | ++++ | ++++ | + | + | +++ | ++++ | ++++ |
| KhES-3 | +++ | +++ | ++++ | ++++ | ++++ | +++ | + | +++ | +++ | ++++ | ++++ |
| H9 | ++++ | +++ | ++++ | ++++ | ++++ | +++ | +/− | +++ | +++ | ++++ | ++++ |
| 2102Ep | +/− | + | ++++ | ++++ | +++ | +++ | + | +/− | +++ | +++ | ++++ |
| NCR-G3 | ++ | + | ++++ | ++++ | ++++ | +++ | ++ | + | ++++ | ++++ | ++++ |

It was found that R-17F antibody strongly binds to human iPS cell and ES cell and scarcely binds to human EC cell (Table 1), similar to R-10G already reported.

R-17F antibody clearly and uniformly stained the whole cellular membrane of almost all human iPS cells (FIG. 2). This staining property was clearly distinguished from SSEA-3, SSEA-4 and the like, which are conventional pluripotent stem cell marker antibodies (FIG. 2, lower panel). That is, while SSEA-3 and SSEA-4 also stained cellular membrane, the staining was not uniform depending on the portion. R-17F antibody was shown to be a novel marker antibody not known heretofore, also from the intracellular localization of the epitope.

### 3. Cytotoxic Activity of R-17F Antibody Against Human iPS Cell

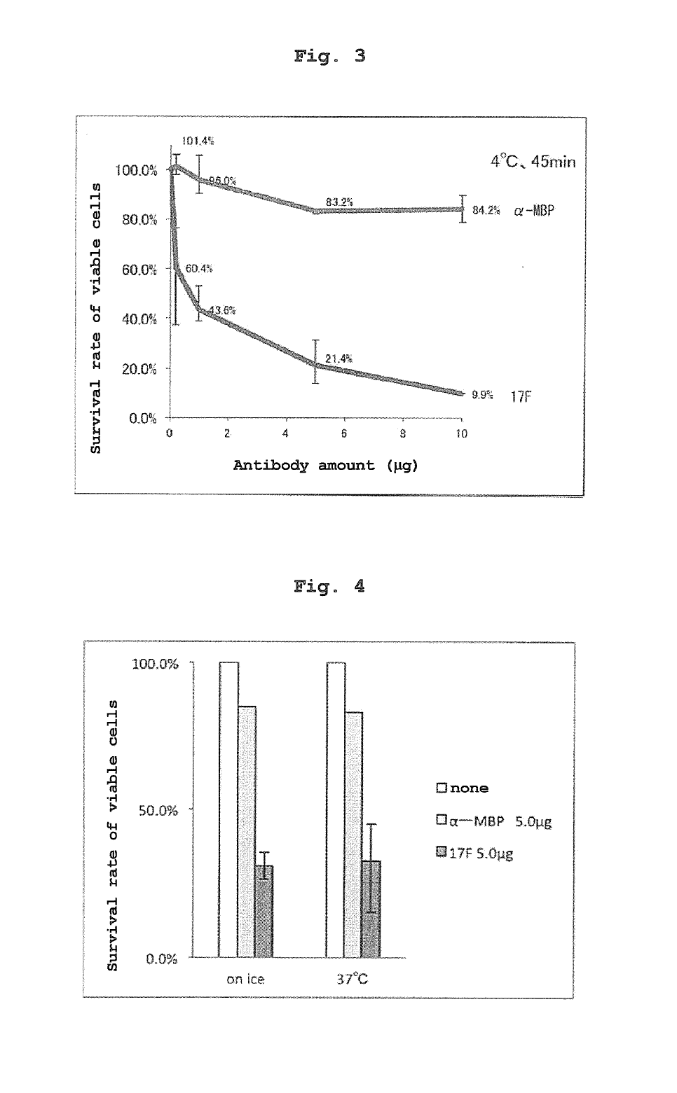

To examine the presence or absence of cytotoxic activity of R-17F antibody against human iPS cells, various concentrations of R-17F antibody were added to Tic cell suspension, and the mixture was reacted at 4° C. for 45 min. 7-AAD that stains only dead cells was added, and the survival rate of the Tic cells was measured by FACS analysis. As a control, anti-mannan-binding protein (MBP) antibody was used. As a result, R-17F antibody showed a concentration-dependent strong cytotoxic activity against human iPS cells (FIG. 3).

To investigate the mechanism of the cytotoxic activity of R-17F antibody, the temperature dependency of the cytotoxic activity was examined. That is, the cell survival rate was measured in the same manner as above for reaction of Tic cells and R-17F antibody for 45 min in ice water, and the reaction at 37° C. As a result, the cytotoxic activity progressed almost the same under the both conditions, which suggests that the cytotoxic action is a reaction independent of complement (enzyme) (FIG. 4).

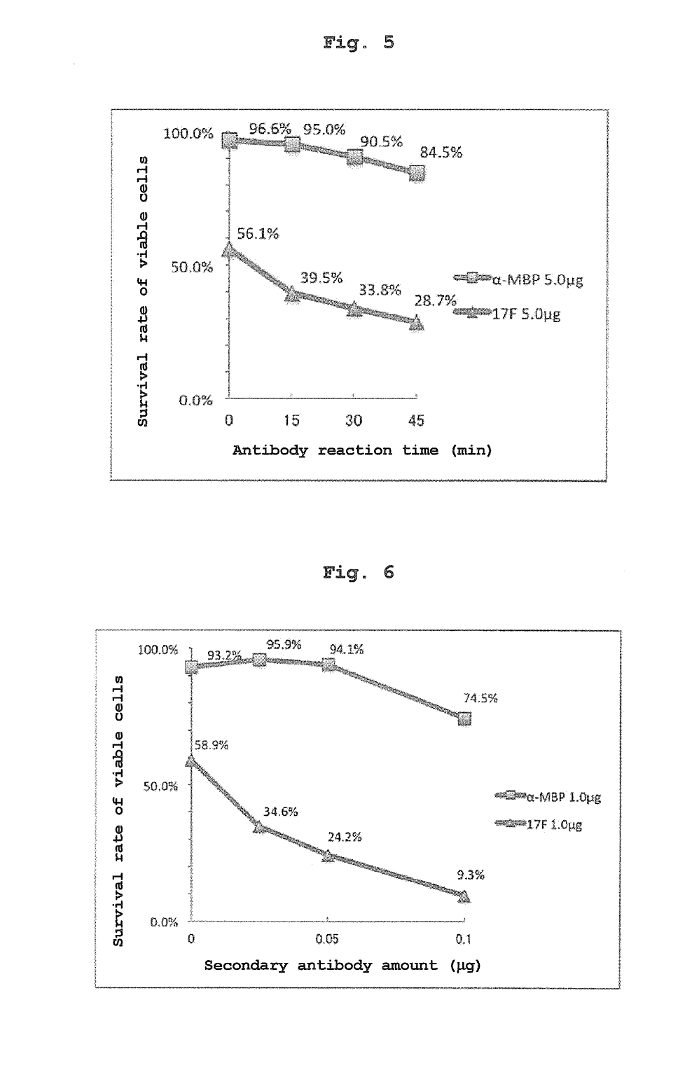

Then, time-course changes of the survival rate of Tic cells by the addition of R-17F antibody were monitored every 15 min up to 45 min. As a result, almost half of Tic cells died immediately after addition of R-17F antibody, and the survival rate also decreased thereafter in a reaction time dependent manner (FIG. 5).

Then, whether addition of a secondary antibody influences the cytotoxic activity of R-17F antibody against human iPS cells was examined. As a result, the cytotoxic activity of R-17F antibody was markedly enhanced by the addition of a very small amount of the secondary antibody (FIG. 6) .

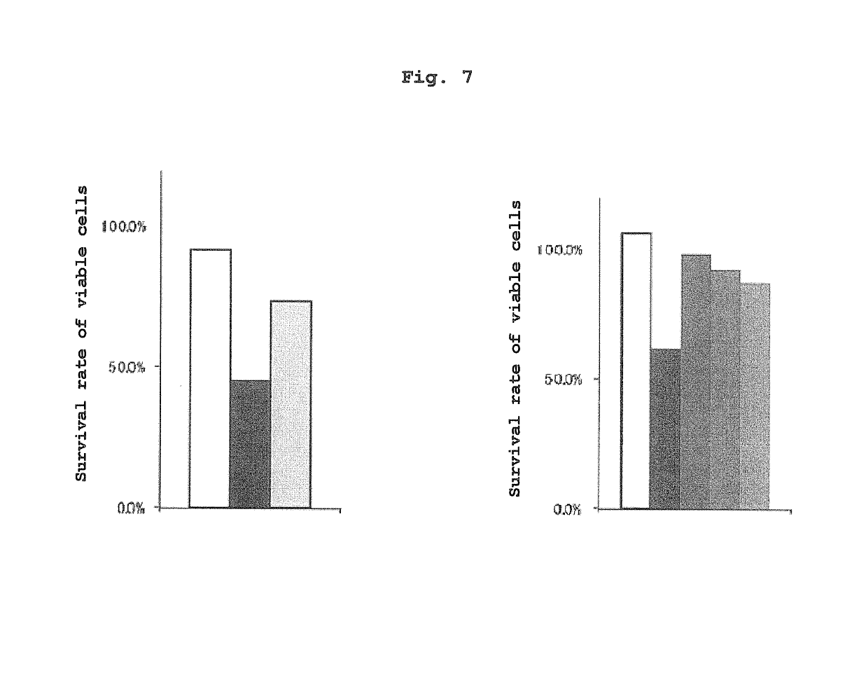

Lastly, the cytotoxic activity of other anti-iPS/ES cell antibodies against human iPS cells was compared to that of R-17F antibody. Anti-MBP antibody was used as a negative control. As a result, none of R-10G already reported by the present inventors, and existing antibodies (TRA-1-60, TRA-1-81, SSEA-4) showed a significant cytotoxic activity (FIG. 7).

From the above, it was confirmed that the cytotoxic activity of R-17F antibody against human iPS cells is a characteristic action not seen in other known anti-iPS/ES cell antibodies.

Furthermore, in human tissues, human normal tissues and fetal tissue array (including cerebrum, cerebellum, heart, stomach, liver, lung, thymus, colon, kidney, spleen, placenta, bladder, skin, muscular tissue, tongue (BioChain Institution, Inc. Hayward, Calif.)) were histochemically studied using a fluorescence-labeled antibody. As a result, exceptionally weak staining was found in 1-2 tissues, and the staining was below detection limit in other tissues.

4. Ubiquitous binding of R-17F antibody to human iPS/ES cell

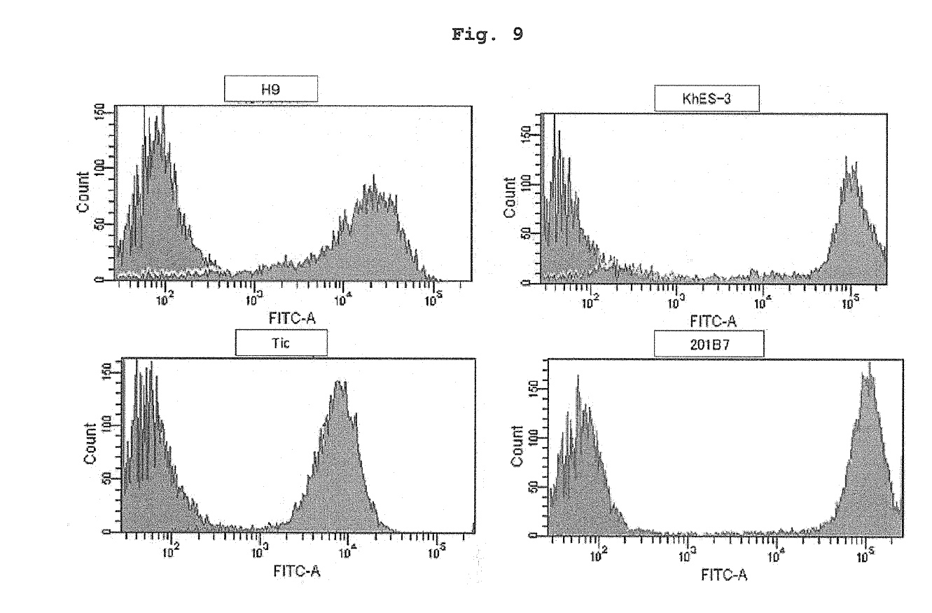

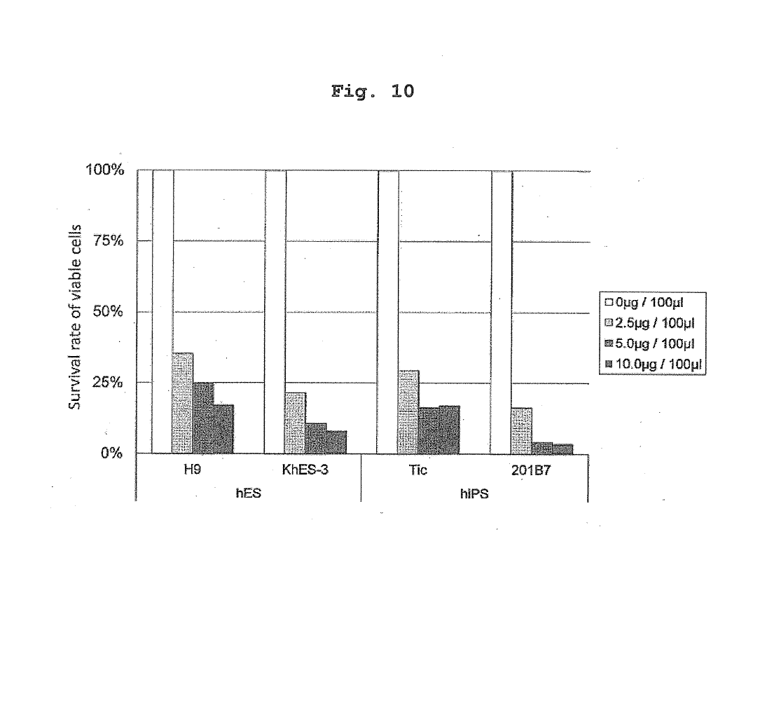

The reactivity of R-17F antibody with human iPS cells (Tic, 201B7) and human ES cells (KhES-3, H9) was quantitatively analyzed by flow cytometry. R-17F antibody showed a single cell peak at a high binding site in four kinds of cell lines of Tic, 201B7 which is one kind of the iPS cell lines produced for the first time in the world by Dr. Yamanaka, and H9 and KhES-3 which are representative human ES cell lines, thus showing that non-uniform binding is less among the cells in the same line (FIG. 9). The result is in conformity with the result of the R-17F antibody staining test using a confocal laser microscope as shown in FIG. 2 that all cells are R-17F antibody positive, and strongly suggests that R-17F antibody has properties of a ubiquitous marker antibody which widely binds to iPS and ES cells in general.

5. Cytotoxic Activity of R-17F Antibody Against Human iPS/ES Cells

The cytotoxic activity of R-17F antibody against human iPS cells (Tic, 201B7) and human ES cells (KhES-3, H9) was analyzed. R-17F antibody was added, reaction was performed at 4° C. for 45 min, 7-AAD that stains only dead cells was added, and the survival rate of the cells was measured by FACS analysis. As a result, R-17F antibody showed an antibody-concentration-dependent cytotoxic activity against all these cell lines (FIG. 10). The sensitivity to the cytotoxic activity of R-17F was not largely different among the cell lines. That is, it was strongly suggested that R-17F antibody has a ubiquitous cytotoxic activity against human iPS/ES cells.

### 6. Suppressive Action of R-17F Antibody on Human iPS Cell Colony Growth

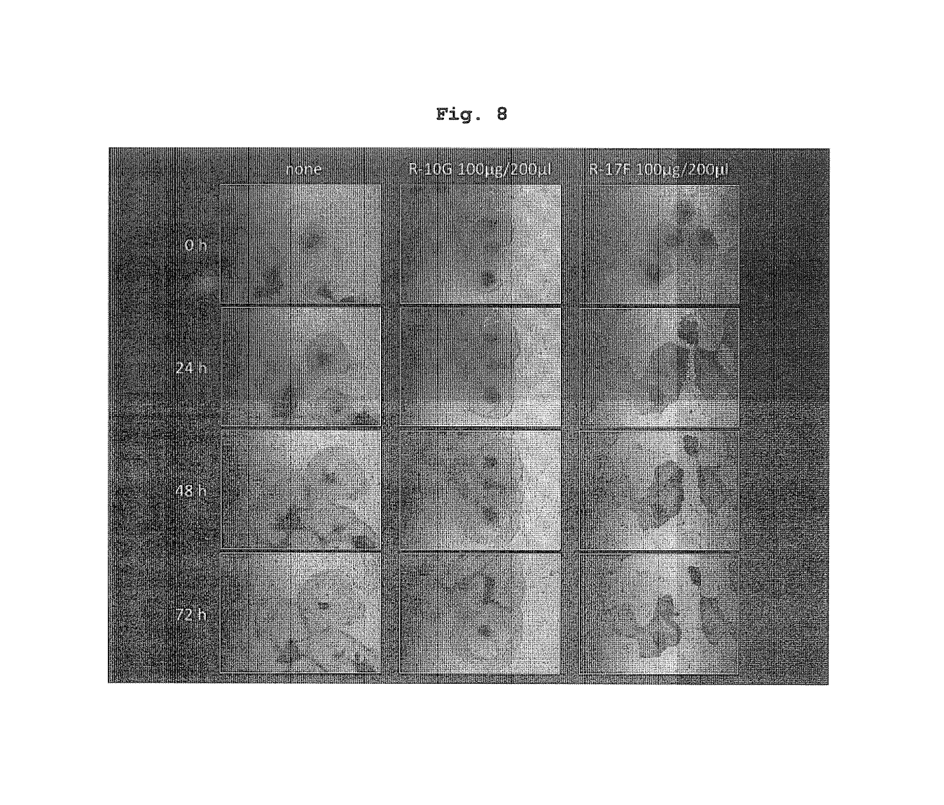

In the study up to this point, the R-17F antibody was strongly suggested to have universal cytotoxic activity colony damage activity against human iPS/ES cells. However, in these studies, the cytotoxic activity of R-17F antibody was assayed by adding R-17F to iPS cells suspension-cultured in a single cell state. In fact, however, iPS cells do not divide and grow in a single cell suspension state, but grow by forming a colony in an adhered state. Thus, if the possibility of utilization of R-17F antibody as a selective elimination agent of human iPS/ES cells in regenerative medicine is considered, it is necessary to examine the effect of R-17F on the growth of iPS cells that formed a colony. Accordingly, the effect of R-17F antibody on the growth of Tic cell colony was examined up to 72 hr of culture. During this period, Tic cell colony grew at a doubling time of about 24 hr. When cultivated with the addition of R-17F antibody, the colony grew slightly in 24 hr, stopped growing in 48 hr, and started to regress to the original colony size or smaller in 72 hr. When R-10G antibody that selectively binds to low sulfated keratin sulfuric acid of human iPS/ES cells was added, colony growth was not influenced at all and a huge colony was grown in 72 hr. Thus, it was shown that R-17F antibody selectively inhibits the growth of Tic cell colony (FIG. 8).

### 7. Isolation and Structural Analysis of R-17F Antibody Epitope

From the results of Western blotting in the above-mentioned 1. (FIG. 1B) and the results of immunostaining of Tic cells (FIG. 2), the present inventors predicted that R-17F antibody might recognize a lipid substance on human iPS/ES cells as an epitope. To verify this hypothesis, Tic cells were first treated with D-PDMP, which is known to inhibit an enzyme reaction converting ceramide to glucosylceramide (GluCer), which is a starting material for the biosynthesis of ganglioside series or globoside series glycolipids, whereby glycolipid expression on a cellular surface was suppressed. The D-PDMP-treated Tic cells were reacted with R-17F antibody, a fluorescence-labeled secondary antibody was added and changes in the reactivity of R-17F antibody with Tic cells was examined by FACS analysis. As a result, the average fluorescence intensity of D-PDMP-treated Tic cells decreased to 48.9% as compared to untreated Tic cells (FIG. 11A). Although not shown in the Figure, SSEA-4 known to recognize glycolipid also showed a similar behavior (decrease to 28.0%) to R-17F antibody, but TRA-1-60 that recognizes glycoprotein did not show changes in the reactivity with Tic cells as a result of the D-PDMP treatment. From the above, a possibility of R-17F antibody recognizing a glycolipid molecule specifically expressed on human iPS/ES cell surface was suggested.

Next, total lipid components were extracted from the cellular membrane of the Tic cells. After TLC separation, it was transcribed onto PVDF membrane, and the reactivity with R-17F antibody was examined by Far-eastern blotting. As a result, the main spot (A) was detected near globoside, and one minor spot was observed thereabove (FIG. 11B). Although not shown in the Figure, these spots were different from those detected using SSEA-4 antibody as a probe.

Then, the lipid fraction of Tic cells was fractionated by TLC, and purification of main spot (A) was tried. The separation conditions by TLC were considered, and separation TLC was performed carefully. As a result, main spot (A) was successfully purified as a mass spectrometrically uniform standard product. That is, the mass spectrum obtained by MALDI-TOF-MS showed an assignable signal in m/z 1000-2000 region, thus indicating that spot A is a glycolipid purified to high purity (FIG. 11C). These signals were subjected to MSⁿ measurement, and the structures were confirmed. By these experiments, the structure of spot A was identified as a glycolipid containing ceramide, Fuc-Hex-HexNAc-Hex-Hex-ceramide. In addition, a sugar residue involved in the activity was identified by treating R-17F positive spot with various glycosidases.

### 8. Analysis of R-17F Antibody Epitope by Sugar Chain Microarray

Based on the results of structural analysis of epitope by mass spectrometry, neoglycolipids obtained by fluorescence labeling lacto series and neolacto series sugar chains with ADHP (N-aminoacetyl-N-(9-antharacenyl methyl)-1,2-dihexadecyl-sn-glycero-3-phosphoethanolamine) were spotted on a nitrocellulose membrane, and the reactivity of R-17F antibody was examined. As a result, a remarkable binding activity was found in LNFP I: Lacto-N-fucopentaose I [Fuc(α1-2)Gal(β1-3)GlcNAc(β1-3)Gal(β1-4)Glc], but binding activity was not found at all in LNnT: Lacto-N-neotetraose [Gal(β1-4)GlcNAc(β1-3)Gal(β1-4)Glc], LNT: Lacto-N-tetraose [Gal(β1-3)GlcNAc(β1-3)Gal(β1-4)Glc, Lewis b: Lacto-N-difucohexose I (LNDFH I) [Fuc(α1-2)Gal(β1-3)[Fuc(α1-4)]GlcNAc(β1-3)Gal(β1-4)Glc, Lewis a: Lacto-N-fucopentaose II (LNFP II) [Gal(β1-3)[Fuc(α1-4)]GlcNAc(β1-3)Gal(β1-4)Glc (FIG. 12). These results show that Fuc(α1-2)Gal(β1-3)GlcNAc structure plays an important role as an R-17F antibody epitope.

### 9. Determination of Base Sequence of Variable Region of R-17F Antibody Gene

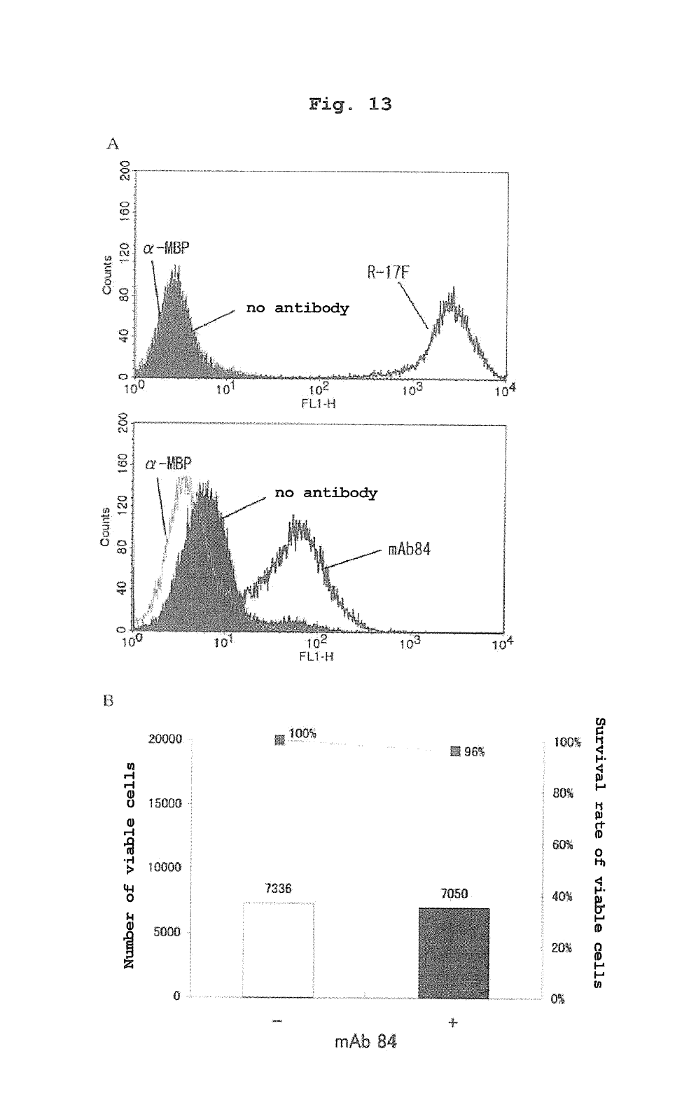

cDNA containing each variable region of heavy chain and light chain was amplified by 5′-RACE PCR using total RNA prepared from hybridoma R-17F. The amplified product was cloned into a plasmid vector, base sequence analysis was performed, and the amino acid sequence to be encoded was assumed from the results of the obtained base sequence (heavy chain base sequence: FIG. 13-A, light chain base sequence: FIG. 13-B). CDR was analyzed using IMGT/V-QUEST (http://www.imgt.org/IMGT_vquest/share/textes/). As a result, CDRs of the heavy chain and light chain were assumed as follows.

heavy chain
CDR 1
(SEQ ID NO: 1)
GFTFSYYW
CDR 2
(SEQ ID NO: 2)
IRLKSDNYAT
CDR 3
(SEQ ID NO: 3)
EGFGY
light chain
CDR 1
(SEQ ID NO: 4)
QDVSTA
CDR 2
(SEQ ID NO: 5)
WAS
CDR 3
(SEQ ID NO: 6)
QQHYSTPRT

10. Comparison with mAb84

To clarify the difference between mAb84 described in WO 2007/102787 and R-17F antibody, the reactivities of the both with human iPS cell Tic were compared by FACS analysis. As a negative control, anti-MBP antibody was used. As a result, the reactivity of mAb84 with the Tic cell was shown to be weaker than that of R-17F antibody (FIG. 13A).

In the same manner as in the above-mentioned 3, the cytotoxic activity of mAb84 against Tic cell was examined. Since addition of mAb84 scarcely decreased the survival rate of Tic cells (FIG. 13B), it was considered that mAb84 does not have a strong cytotoxic activity against human IPS cell such as that of R-17F antibody and the like. In this respect, additional experiments were performed thereafter as regards cell tissue staining, flow cytometry analysis, cytotoxic action and the like. However, the results were poor in reproducibility and, in some cases, bindability and cytotoxic activity of the same level as those of R-17F antibody were observed for human iPS cells. The reason for low reproducibility is unclear at present.

mAb84 is an antibody recognizing human podocalyxin-like protein I, and the subtype is IgM. R-17F antibody is an antibody that recognizes glycolipids, and the subtype is IgG1. The amino acid sequences of CDR1-CDR3 of the heavy chain and light chain of the both do not show homology.

### REFERENCE DOCUMENTS

1. Choo A B, Tan H L, Ang S N et al. Selection against undifferentiated human embryonic stem cells by a cytotoxic antibody recognizing podocalyxin-like protein-1. Stem Cells 2008; 26:1454-1463.
2. Furue M, Okamoto T, Hayashi Y et al. Leukemia inhibitory factor as an anti-apoptotic mitogen for pluripotent mouse embryonic stem cells in a serum-free medium without feeder cells. In vitro Cell Dev Biol Anim 2005; 41:19-28.
3. Furue M K, Na J, Jackson J P et al. Heparin promotes the growth of human embryonic stem cells in a defined serum-free medium. Proc Natl Acad Sci USA 2008; 105:13409-13414.
4. Takahashi K, Tanabe K, Ohnuki M et al. Induction of pluripotent stem cells from adult human fibroblasts by defined factors. Cell 2007; 131:861-872.
5. Toyoda M, Yamazaki I M, Itakura Y et al. Lectin microarray analysis of pluripotent and multipotent stem cells. Genes to Cells 2011; 16:1-11.
6. Taki, T., Ishikawa, D., TLC blotting: application to microscale analysis of lipids and as a new approach to lipid-protein interaction, Anal Biochem 251 (1997) 135-143.
7. Watanabe K, Ueno M, Kamiya D et al. A ROCK inhibitor permits survival of dissociated human embryonic stem cells. Nat Biotechnol 2007; 25:681-686.

While the present invention has been described with emphasis on preferred embodiments, it is obvious to those skilled in the art that the preferred embodiments can be modified. The present invention intends that the present invention can be embodied by methods other than those described in detail in the present specification. Therefore, the present invention encompasses all modifications encompassed in the gist and scope of the appended “CLAIMS.”

The contents disclosed in any publication cited herein, including patents and patent applications, are hereby incorporated in their entireties by reference, to the extent that they have been disclosed herein.

This application is based on a patent application No. 2012-280259 filed in Japan (filing date: Dec. 21, 2012), the contents of which are incorporated in full herein.

### INDUSTRIAL APPLICABILITY

The anti-iPS/ES cell antibody of the present invention is significant as a human iPS/ES cell positive and EC cell negative novel monoclonal antibody, since it has added a new index for setting the standard for (standardization of) human iPS/ES cell. Furthermore, the antibody of the present invention having a target cell specific cytotoxic activity is considered to have an important meaning in regenerative medicine utilizing pluripotent stem cells, and is highly useful for the preparation of safe cells and tissues for transplantation into human.

1.-18. (canceled)

19. A monoclonal IgG antibody that recognizes a glycolipid on an iPS cell and ES cell surface as an epitope, does not recognize EC cells, and has a cytotoxic activity against a target cell, wherein the epitope comprises a sugar chain represented by the following formula:
Fuc-Hex-HexNAc-Hex-Hex
wherein Fuc is fucose, Hex is hexose, and HexNAc is N-acetylhexosamine, excluding a monoclonal antibody produced by hybridoma R-17F (accession number: NITE BP-01425).

20. The antibody according to claim 19, wherein the iPS and ES cells are derived from human.

21. The antibody according to claim 19, which recognizes, as an epitope, at least a region comprising a sugar chain represented by the following formula:
Fuc(α1-2)Gal(β1-3)GlcNAc(β1-3)Gal(β1-4)Glc
wherein Fuc is fucose, Gal is galactose, GlcNAc is N-acetylglucosamine, and Glc is glucose, in the glycolipid.

22. The antibody according to claim 19 comprising
(a) CDR comprising the amino acid sequence shown in SEQ ID NO: 1,
(b) CDR comprising the amino acid sequence shown in SEQ ID NO: 2,
(c) CDR comprising the amino acid sequence shown in SEQ ID NO: 3,
(d) CDR comprising the amino acid sequence shown in SEQ ID NO: 4,
(e) CDR comprising the amino acid sequence shown in SEQ ID NO: 5, and
(f) CDR comprising the amino acid sequence shown in SEQ ID NO: 6.

23. The antibody according to claim 19, comprising (1) a heavy chain variable region comprising the amino acid sequence shown in SEQ ID NO: 8, and
(2) a light chain variable region comprising the amino acid sequence shown in SEQ ID NO: 10.

24. A reagent for detecting an iPS or ES cell, comprising the antibody according to claim 19.

25. A method of detecting an iPS or ES cell, comprising contacting a cell sample with the antibody according to claim 19, and detecting a cell bound to the antibody in the sample.

26. An agent for eliminating an iPS or ES cell, comprising the antibody according to claim 19.

27. The agent according to claim 26, further comprising a secondary antibody to the aforementioned antibody.

28. An agent for a cell transplantation therapy, comprising a cell population differentiated from iPS or ES cells and the antibody according to claim 19.

29. A method of eliminating an iPS or ES cell in a cell population, comprising contacting the cell population with a monoclonal IgG antibody that recognizes a glycolipid on an iPS cell and ES cell surface as an epitope, does not recognize EC cells, and has a cytotoxic activity against a target cell, wherein the epitope comprises a sugar chain represented by the following formula:
Fuc-Hex-HexNAc-Hex-Hex
wherein Fuc is fucose, Hex is hexose, and HexNAc is N-acetylhexosamine.

30. The method according to claim 29, comprising further contacting the cell population with a secondary antibody to the aforementioned antibody.

31. A method of producing a uniform differentiated cell population free of an undifferentiated cell, comprising contacting a cell population differentiated from an iPS or ES cell with a monoclonal IgG antibody that recognizes a glycolipid on an iPS cell and ES cell surface as an epitope, does not recognize EC cells, and has a cytotoxic activity against a target cell, wherein the epitope comprises a sugar chain represented by the following formula:
Fuc-Hex-HexNAc-Hex-Hex
wherein Fuc is fucose, Hex is hexose, and HexNAc is N-acetylhexosamine, and recovering viable cells.

32. The method according to claim 31, comprising further contacting the cell population differentiated from the aforementioned iPS or ES cell with a secondary antibody to the aforementioned antibody.

33. An agent for a cell transplantation therapy, comprising a differentiated cell population obtained by the method according to claim 31.
